(19) United States
(12) Patent Application Publication
(10) Pub. No.: US 2024/0254555 A1
(43) Pub. Date: Aug. 1, 2024

(54) Heart Transplant Rejection mRNA prognostic and diagnostic Biomarkers

(71) Applicant: THE TRUSTEES OF THE UNIVERSITY OF PENNSYLVANIA, Philadelphia, PA (US)

(72) Inventors: Brendan Keating, Philadelphia, PA (US); Bao-Li Chang, Paoli, PA (US); Abraham Shaked, Wynnewood, PA (US)

(21) Appl. No.: 18/427,192

(22) Filed: Jan. 30, 2024

Related U.S. Application Data
(60) Provisional application No. 63/482,278, filed on Jan. 30, 2023.

(51) Int. Cl. C12Q 1/6883; C12Q 1/6874

(52) U.S. Cl. CPC C12Q 1/6883; C12Q 1/6874; C12Q 2600/118; C12Q 2600/158

(57) ABSTRACT

The present invention provides compositions, and methods useful for the diagnosis and treatment of acute rejection of cardiac transplant tissue in subjects in need thereof.

### CROSS-REFERENCE TO RELATED APPLICATION

The present application claims priority under 35 U.S.C. § 119(e) to U.S. Provisional Patent Application No. 63/482,278, filed Jan. 30, 2023, which is hereby incorporated by reference in its entirety herein.

### STATEMENT REGARDING FEDERALLY SPONSORED RESEARCH AND DEVELOPMENT

This invention was made with government support under AI144522 and AI063589 awarded by the National Institutes of Health. The government has certain rights in the invention.

### BACKGROUND OF THE INVENTION

Heart transplantation provides a significant improvement in survival and quality of life for patients with end-stage heart disease, however many recipients experience different levels of graft rejection that can be associated with significant morbidities and mortality. Current clinical standard-of-care for the evaluation of heart transplant acute rejection (AR) consists of routine endomyocardial biopsy (EMB) followed by visual assessment by histopathology for immune infiltration and cardiomyocyte damage. These techniques are often not optimal for the early detection of rejection due to observer variation and the fact that rejection processes may be advanced by the time they are visibly by histology.

Thus, a need exists in the art for methods and compositions which are able to easily perform molecular profiling in post-transplant cardiac tissue to monitor for expression of genes associated with the early phases of acute transplant rejection, so that effective treatment can be imitated sooner. The current invention addresses these needs.

### SUMMARY OF THE INVENTION

As described herein the current invention relates to compositions, and methods useful for the diagnosis and treatment of acute rejection of cardiac transplant tissue in subjects in need thereof.

In one aspect, disclosed herein is a method for diagnosing and treating acute rejection (AR) of a cardiac transplant tissue in a subject, the method comprising:
contacting a biological sample from the subject with a reagent for assaying the level of at least one biomarker signature nucleic acid,
detecting the amount of the at least one biomarker signature nucleic acid in the biological sample using the reagent,
comparing the level of the at least one biomarker signature nucleic acid from the biological sample to the level of the at least one biomarker signature nucleic acid from a control sample,
determining whether the level of the at least one biomarker signature nucleic acid in the biological sample is an equivalent level, an increased level or a decreased level of the at least one biomarker signature nucleic acid compared to the control sample, wherein an increased level or a decreased level of the at least one biomarker signature nucleic acid in the biological sample relative to the level of the at least one biomarker signature nucleic acid from the control sample indicates that the subject has or is at risk of acute rejection of the cardiac transplant tissue,
and, when the subject is determined to have or be at risk of acute rejection of the cardiac transplant tissue, recommending or providing a treatment to the subject thereby treating the acute rejection.

In certain embodiments, the at least one biomarker signature nucleic acid is a transcript of a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, ZCCHC12, and any combination thereof.

In certain embodiments, the at least one biomarker signature nucleic acid is RNA.

In certain embodiments, the detecting the amount of the at least one biomarker signature nucleic acid in the biological sample is performed using RNA-seq.

In certain embodiments, the biological sample is an endomyocardial biopsy.

In certain embodiments, the biological sample is peripheral blood.

In certain embodiments, the treatment is selected from the group consisting of increasing the dose or frequency of administration of one or more rejection-ameliorating medications, changing to a different rejection-ameliorating medication, and administering one or more medications that suppress the immune system.

In certain embodiments, the rejection-ameliorating medication is selected from the group consisting of tacrolimus, cyclosporine, prednisone, mycophenolate, azathioprine, sirolimus, everolimus, and any combination thereof.

In certain embodiments, the subject is a human.

In another aspect, the disclosure provides a method for determining whether a subject is at risk for developing acute rejection (AR) of a cardiac transplant tissue and then treating the subject therefor, the method comprising:
contacting a biological sample from the subject with a reagent for assaying the level of at least one biomarker signature nucleic acid is a transcript of a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, and ZCCHC12,
detecting the amount of the at least one biomarker signature nucleic acid in the biological sample using the reagent,
comparing the level of the at least one biomarker signature nucleic acid from the biological sample to the level of the at least one biomarker signature nucleic acid from a control sample, and
determining whether the level of the at least one biomarker signature nucleic acid in the biological sample is an equivalent level, an increased level or a decreased level of the at least one biomarker signature nucleic acid compared to the control sample, wherein an increased level or a decreased level of the at least one signature nucleic acid in the biological sample relative to the level of the at least one biomarker signature nucleic acid from the control sample indicates that the subject is at risk for developing the acute rejection,
and, when the subject is at risk of developing the acute rejection of a cardiac transplant, recommending or providing a treatment to the subject.

In certain embodiments, the nucleic acid is RNA.

In certain embodiments, the detecting of the at least one biomarker signature nucleic acid in the biological sample is performed using RNA-seq.

In certain embodiments, the biological sample is an endomyocardial biopsy.

In certain embodiments, the biological sample is peripheral blood.

In certain embodiments, the treatment is selected from the group consisting of increasing the dose or frequency of administration of one or more rejection-ameliorating medications, changing to a different rejection-ameliorating medication, and administering one or more medications that suppress the immune system.

In certain embodiments, the rejection-ameliorating medication is selected from the group consisting of tacrolimus, cyclosporine, prednisone, mycophenolate, azathioprine, sirolimus, everolimus, and any combination thereof.

In another aspect, the disclosure provides a composition comprising reagents for assaying the level of at least two biomarker signature nucleic acids useful for diagnosing, predicting, and/or monitoring acute rejection of a cardiac transplant tissue in a sample of a subject, wherein the at least two biomarker signature nucleic acids are transcripts of a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, ZCCHC12, or fragments, or variants thereof.

In certain embodiments, the biomarker signature nucleic acids are RNA.

In another aspect, the disclosure provides a kit or assay device for use in diagnosing acute rejection of a cardiac transplant tissue, the kit comprising the composition of any of the above aspects or embodiments, or any aspect or embodiment disclosed herein and instructions for use.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1C illustrate the gene expression differences between rejection and non-rejection specimens. FIG. 1A) Heatmap of differentially expressed genes between AR and non-rejection tissues. FIG. 1B) Volcano plot for the AR and NR comparison. FIG. 1C) Violin plots for selected differentially expressed genes between AR and NR.

FIGS. 2A-2B illustrate a pathway assessment of AR versus NR genes. FIG. 2A) KEGG pathway enrichment for AR versus NR genes. FIG. 2B) Gene Ontology enrichment for AR versus NR differentially expressed genes.

FIG. 3A-3B illustrate the clinical utility of acute rejection genes. FIG. 3A). ROC curve for the AR signature as a classifier between rejection and nonrejection biopsies with calculated AUC. FIG. 3B) Performance statistics for Random Forest classification using the AR signature. Abbreviated metrics correspond to the following: positive predictive value (PPV), negative predictive value (NPV), false positive rate (FPR), false negative rate (FNR), false discovery rate (FDR) and Matthews correlation coefficient (MCC).

FIGS. 4A-4B illustrate that characterization of non-rejection biopsies: preceding rejection versus preceding non-rejection. FIG. 4A) Volcano plot of gene expression differences for PR vs PNR biopsies. FIG. 4B) Volcano plots of top differentially expressed genes between PR and PNR.

FIGS. 5A-5D illustrate the functional characterization of biopsies preceding rejection. FIG. 5A) KEGG pathway enrichment for PR versus PNR gene expression. FIG. 5B) GO pathway enrichment for PR versus PNR biopsies. FIG. 5C) ROC curve for the PR classifier. FIG. 5D) Performance statistics for the PR classifier.

FIGS. 6A-6B illustrate the further characterization of acute rejection transcriptional profiles. FIG. 6A) Overlap of the AR signature with the prior AMR signature identified by Loupy et al. FIG. 6B) Overlap of the AR signature with the TCMR and ABMR signatures identified by Xiu et al.

FIGS. 7A-7B illustrate the gene expression differences between post AR and non-post AR non-rejection biopsies. FIG. 7A) Volcano plot showing genes differentially expressed between post-AR 0R/1R biopsies and non-post-AR 0R/1R biopsies. FIG. 7B) KEGG pathway differences between post-AR biopsies and non-post-AR biopsies.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only and is not intended to be limiting.

The articles “a”, “an”, and “the” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “biomarker” or “marker” as used herein generally refers to a nucleic acid molecule, clinical indicator, protein, or other analyte that is associated with a disease. In various embodiments, a marker is differentially present in a biological sample obtained from a subject having or at risk of developing a disease (e.g., a transplant-related disease) relative to a reference. A marker is differentially present if the mean or median level of the biomarker present in the sample is statistically different from the level present in a reference. A reference level may be, for example, the level present in an environmental sample obtained from a clean or uncontaminated source. A reference level may be, for example, the level present in a sample obtained from a healthy control subject or the level obtained from the subject at an earlier timepoint, i.e., prior to treatment or transplant. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing a disease (e.g., a transplant-related disease), for determining the prognosis of the subject, for evaluating therapeutic efficacy, or for selecting a treatment regimen.

By the term “biomarker signature” is meant a group of one or more biomarkers associated with a particular disease or condition, or the likelihood of a subject to develop the disease or condition.

By “biomarker signature gene product” is meant the nucleic acid (e.g., RNA) transcript or translated polypeptide product of a biomarker signature gene. Biomarker signature gene products may be coding or non-coding RNAs, including messenger RNAs (mRNAs), pre-mRNAs, microRNAs (miRNAs), and the like.

By “agent” is meant any nucleic acid molecule, small molecule chemical compound, antibody, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

The term “co-activator” refers to a protein that binds indirectly to DNA that positively regulates gene expression.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

By “fragment” is meant a portion of a nucleic acid or polypeptide molecule. This portion contains, preferably, at least 10%, 20%, 30%, 40%, 50%, 60%, 70%, 80%, or 90% of the entire length of the reference nucleic acid molecule or polypeptide. A fragment may contain 5, 10, 15, 20, 30, 40, 50, 60, 70, 80, 90, 100 or more nucleotides or amino acids.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. In some cases, homology can also be defined as analogous subunit positions in two molecules, such as polypeptides, having biochemically similar residues (e.g., a serine and/or a threonine, as both have polar and uncharged side chains). The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Hybridization” means hydrogen bonding, which may be Watson-Crick, Hoogsteen or reversed Hoogsteen hydrogen bonding, between complementary nucleobases. For example, adenine and thymine are complementary nucleotides that pair through the formation of hydrogen bonds.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “marker profile” is meant a characterization of the signal, level, expression or expression level of two or more markers (e.g., polynucleotides).

By the term “microbe” is meant any and all organisms classed within the commonly used term “microbiology,” including but not limited to, bacteria, viruses, fungi and parasites.

By the term “microarray” is meant a collection of nucleic acid probes immobilized on a substrate. As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring. Nucleic acid molecules useful in the methods of the invention include any nucleic acid molecule that specifically binds a target nucleic acid (e.g., a nucleic acid biomarker). Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Parenteral” administration of a composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By “reference” is meant a standard of comparison. As is apparent to one skilled in the art, an appropriate reference is where an element is changed in order to determine the effect of the element. In one embodiment, the level of a target nucleic acid molecule present in a sample may be compared to the level of the target nucleic acid molecule present in a clean or uncontaminated sample. For example, the level of a target nucleic acid molecule present in a sample may be compared to the level of the target nucleic acid molecule present in a corresponding healthy cell or tissue or in a diseased cell or tissue (e.g., a cell or tissue derived from a subject having a disease, disorder, or condition).

As used herein, the term “sample” includes a biologic sample such as any tissue, cell, fluid, or other material derived from an organism.

By “specifically binds” is meant a compound (e.g., nucleic acid probe or primer) that recognizes and binds a molecule (e.g., a nucleic acid biomarker), but which does not substantially recognize and bind other molecules in a sample, for example, a biological sample.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, and more preferably more, such as 80% or 85%, and more preferably 90%, 95%, 96%, 97%, 98%, or even 99% or more identical at the amino acid level or nucleic acid to the sequence used for comparison.

Sequence identity and homology is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs).

Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹¹¹ indicating a closely related sequence. In another exemplary approach, a BLOSOM substitution matrix may be used to score conservative and/or non-conservative substitutions.

By “subject” is meant a mammal, including, but not limited to, a human or non-human mammal, such as a bovine, equine, canine, ovine, feline, mouse, or monkey. The term “subject” may refer to an animal, which is the object of treatment, observation, or experiment (e.g., a patient).

By “target nucleic acid molecule” is meant a polynucleotide to be analyzed. Such polynucleotide may be a sense or antisense strand of the target sequence. The term “target nucleic acid molecule” also refers to amplicons of the original target sequence. In various embodiments, the target nucleic acid molecule is one or more nucleic acid biomarkers.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for the detection or diagnosis of cardiac allograft rejection. In certain embodiments, the compositions and methods comprise biomarker nucleic acid expression signatures determined by the expression of genes related to allograft rejection in endomyocardial biopsy (EMB) tissue specimens. The invention also provides kits useful for aiding in the diagnosis of rejection, including acute rejection, in subjects in need thereof.

### Cardiac Transplant Rejection

Over the last 3 decades advances in immunosuppression therapies and patient management have yielded substantial gains in survival rates for allogenic heart transplant recipients, however, 5-year survival rates remain at only about 74%. One of the major obstacles to extending short- and long-term allograft survival is a lack of robust biomarkers that can be sampled via minimally invasive means in order to diagnose and prognosticate acute rejection (AR) early enough to prevent irreversible damage to the allograft.

While there have been significant advances in noninvasive cell-free nucleic acid-based diagnostics including several large-scale evaluations by the CARGO and GRAfT teams, the current diagnostic standard for AR following cardiac transplantation still entails histopathological evaluation of the allograft by endomyocardial biopsy (EMB) using international standards such as The International Society for Heart & Lung Transplantation (ISHLT) 2013 grading system. While these highly invasive procedures have become safer and more standardized in the last few decades, procedural risks still remain, and interobserver variability in EMB readings greatly impact interpretation. Furthermore, as an individual's immune response is dynamic over time, successive biopsies are needed to capture anti-allograft immunity. A fundamental limitation of for-cause biopsies, which is a biopsy performed when a patient clinically manifests symptoms of rejection, is that allograft injury and irreversible damage may already have occurred, and patients who develop acute rejection (AR) are at higher risk of developing chronic allograft vasculopathy (CAV) which can progress to a number of comorbidities including allograft loss.

In certain aspects, the current invention provides advanced statistical analysis and machine learning techniques to evaluate the gene expression profiles of tissue specimens from heart transplant recipients suspected of having or being at risk for rejection, including acute rejection. The expression pattern of genes associated with rejection are used as biomarkers to create a gene expression signature that can be compared to known standard expression signatures established from normal subjects and transplant recipients not experiencing rejection in order to aid in the rapid diagnosis of the subjects without needing extensive, subjective, and invasive traditional testing methods. In certain embodiments, the biomarker signature of the subjects can inform the types of treatment selected and monitor the efficacy of on-going treatment regimens, as well as provide early warning as to the development of rejection.

### Biomarker Signature Nucleic Acids for Identifying Transplant Rejection

As described herein, the disclosure of the present invention provides biomarker signature genes or gene products (e.g., nucleic acids) whose expression levels can be used to diagnose and/or predict whether a subject has or is at risk of developing transplant rejection including acute rejection, and/or monitor the efficacy of a treatment regimen for transplant rejection. Thus, in one embodiment, the present disclosure relates to biomarker signature genes or gene products (e.g., nucleic acids) that are reliably associated with transplant rejection. In certain embodiments, the signature genes or gene products are nucleic acids, including RNAs, proteins, and the like. These biomarker signature gene products may be used to determine whether a subject, e.g., with or without clinical signs or symptoms of transplant rejection, has, or is at risk of developing transplant rejection or acute rejection. These biomarker signature gene products may also be used to monitor the therapeutic efficacy of a rejection reducing or preventing treatment regimen.

In certain embodiments, the signature genes are selected from one or more of the following: ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, ZCCHC12, or any combination thereof.

In certain embodiments, a subject having or is at risk of developing acute rejection of heart transplant tissue or having or at risk of developing acute rejection may be identified by determining the relative level of a biomarker signature gene products, or a group of biomarker signature gene products, in a sample from the subject, wherein the biomarker signature gene product, or group of biomarker signature gene products, is at least one or more signature genes selected from the group consisting of ABHD16A, ADGRG7, AICDA, ALB, ANKS1B, ARC, BAG6, BRD2, BTLA, C4A, CCL19, CCR6, CCR8, CD3G, CD5, CD72, CDH4, CELF4, CLC, CLNK, CXCL8, CXCR3, EDNRB, FFAR2, FLOT1, FLOT1, FOSB, GNL1, H3P6, HLA-A, HLA-DMB, HLA-DMB, HLA-DMB, HLA-DOA, HLA-DOA, HLA-DOB, HLA-DOB, HLA-DPB1, HLA-DQA1, HLA-DQA2, HLA-DQB1, HLA-DQB1, HLA-DQB1, HLA-DRA, HLA-L, HMGN1P14, HS6ST3, IGHA1, IGHG3, IGHG4, IGHV1-2, IGHV1-24, IGHV1-3, IGHV1-45, IGHV3-11, IGHV3-33, IGHV3-53, IGHV3-72, IGHV4-28, IGHV4-31, IGHV4-55, IGKV1-12, IGKV1-13, IGKV2-28, IGKV2-30, IGKV2D-29, IGKV2D-40, IGKV3D-15, IGLV2-34, IGLV3-1, IGLV3-10, IGLV3-21, ITGB1P1, KCNH7, LAMP3, LEF1, LHFPL5, LRTM1, LY6G5B, MINAR2, MS4A3, MSX1, MTHFD2P1, MTRNR2L4, NAPSB, NOMO1, PITX1, PLA2G2A, PLA2G2D, PPM1N, PPP1R11, PSG2, PSMB8, RNA5-8SP6, RPS3AP46, RPS6P26, SCUBE1, SLC22A9, SLC2A3P1, SPP1, TAP1, TIFAB, TNXB, TRIM26, TSPY3, UBD, UBD, XCR1, ZCCHC12, and any combination thereof.

In certain embodiments, a subject having or is at risk of developing rejection of heart transplant tissue or having or at risk of developing rejection may be identified by determining the relative level of a biomarker signature gene products, or a group of biomarker signature gene products, in a sample from the subject, wherein the biomarker signature gene product, or group of biomarker signature gene products, is at least one or more signature genes selected from the group consisting of ABHD16A, ADAMDEC1, ADGRG7, ADIPOQ, ALB, ARC, BAG6, BRD2, CIDEA, CIDEC, CXCL11, CXCL9, DOC2B, DUX4, DUX4L18, DUX4L19, DUX4L26, FAM220A, FLOT1, FOSB, H3P6, HLA-A, HLA-A, HLA-C, HLA-DMB, HLA-DMB, HLA-DPB1, HLA-DRA, HS6ST2, IDO1, IGHA1, IGHG2, IGHV1-45, IGHV3-15, IGHV3-20, IGHV3-7, IGKV1-16, IGKV1-17, IGLV1-44, IGLV2-14, IGLV7-46, ITGB1P1, KCNJ10, LGALS17A, LHFPL5, MMP7, MTRNR2L1, MTRNR2L11, MTRNR2L8, NA, NOMO1, NPY2R, NWD2, PPP1R11, PPP1R11, PPT2, PSMB8, RNA5-8SP6, SPP1, SRP9P1, TM4SF19, TRAV6, UBD, UBD, UBE2T, and any combination thereof.

Determining whether a level of a signature gene product in a biological sample derived from a subject is different from a level of the biological signature gene product(s) present in a control (e.g., a healthy control or a non-rejection transplant control) subject may be determined by comparing the expression level of the biological signature gene product(s) in the sample from the subject with a suitable control of the same biomarker signature gene products(s). The skilled artisan can select an appropriate control for the assay in question.

Generally, a suitable control may also be a reference standard. A reference standard serves as a reference level for comparison, such that biological samples can be compared to the reference standard in order to infer the development of transplant rejection or to infer the risk of developing or progression of transplant rejection in a subject. A reference standard may be representative of the level of one or more biomarker signature gene product(s) in a known subject, e.g., a subject known to be a normal subject or a transplant recipient subject not experiencing rejection. Likewise, a reference standard may be representative of the level of one or more biomarker signature gene product(s) in a population of known subjects, e.g., a population of subjects known to be normal subjects, or in an alternative embodiment, a population of subjects known to have transplant rejection or a certain type of transplant rejection (e.g., acute rejection).

The biomarker signature gene products described herein can be used individually or in any combination in methods to identify a subject who has or is at risk of developing transplant rejection (e.g., diagnostic tests). Based on the identification of a subject who has transplant rejection, or a subject who is at risk of developing transplant rejection, or a subject who has an early-phase transplant rejection (e.g. 0R/1R) and is at risk of progressing to an acute rejection phase (e.g. 2R/3R), additional procedures may be indicated, including, for example, additional diagnostic tests or therapeutic procedures. The biomarker signature gene products described herein can also be used individually or in any combination in methods to monitor the therapeutic efficacy of a transplant rejection prevention or treatment regimen in a subject who has or is at risk of developing transplant rejection.

### Biological Samples

The expression level of one or more biomarker signature gene products may be determined in a biological sample obtained from or derived from a subject. Such a sample may be further processed after it is obtained from the subject. For example, proteins or nucleic acids (e g., mRNA) may be isolated from a sample. In certain embodiments, the signature gene product may be detected in a sample obtained from a subject non-invasively (e.g., saliva, urine, or the like) or with minimal invasiveness (e.g., via peripheral intravenous blood draw). In certain embodiments, the signature gene product may be detected in a biopsy sample, including endomyocardial biopsy (EMB) taken from the transplant tissue before or after implantation into the subject. In other embodiments, the biological sample is a bodily fluid, for example, a blood sample, or a fraction thereof, a serum sample, a plasma sample, a lymph sample, a urine sample, a saliva sample, a tear sample, a sweat sample, a semen sample, a vaginal sample, a bronchial sample, a mucosal sample, or a cerebrospinal fluid (CSF) sample. It is also contemplated that a series of biological samples can be collected from the subject at different time points (e.g., before and after transplantation) in order to monitor for changes in biomarker signature gene product expression.

### Readouts of Biomarker Signature Gene Expression

In some embodiments, the current invention provides methods for determining the expression levels of transplant rejection-related biological signature genes in a particular tissue sample. In some embodiments, the sample is a biological tissue sample that, for example, derives from a biopsy or formalin-fixed, paraffin-embedded (FFPE) specimen. In certain embodiments, biological samples also include body fluids (e.g., blood, serum, plasma, amniotic fluid, sputum, urine, cerebrospinal fluid, lymph, tear fluid, feces, or gastric fluid), tissue extracts, and culture media (e.g., a liquid in which a cell or tissue, has been grown). In certain embodiments, the biological sample is purified prior to detection using any standard method typically used for isolating nucleic acid molecules (e.g., RNA molecules) from a biological sample.

In some embodiments, the determination of biological signature gene expression can be accomplished by means for analyzing multiple types of nucleic acids present in a sample, including DNA and RNA. In various embodiments, sample preparation involves extracting a mixture of nucleic acid molecules (e.g., DNA and RNA) from the sample.

The expression levels of biological signature genes can be detected by any suitable method. The methods described herein can be used individually or in combination for a more accurate detection of the biological signature genes or gene products. Methods for conducting polynucleotide hybridization assays have been developed in the art. Hybridization assay procedures and conditions will vary depending on the application and are selected in accordance with the general binding methods known including those referred to in: Sambrook and Russell, Molecular Cloning: A Laboratory Manual (3^{rd} Ed. Cold Spring Harbor, N.Y, 2001); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623. A data analysis algorithm (E-predict) for interpreting the hybridization results from an array is publicly available (see Urisman, 2005, Genome Biol 6:R78).

In certain embodiments, the detection and measurement of biological signature gene products is carried out by RNA-seq. RNA-seq, also called “RNAseq,” is a form of whole transcriptome shotgun sequencing (WTSS) technology that utilizes the capabilities of next-generation sequencing (NGS) to reveal a snapshot of total RNA presence and quantity from a transcriptome in one or more cells at a given moment in time. The introduction of high-throughput RNA-seq technologies has greatly aided transcriptomics by allowing RNA analysis through cDNA sequencing at massive scale. NGS platforms used for RNA-seq are well known in the art and are commercially available. In certain embodiment, the whole transcriptome sequencing typically comprises whole mRNA sequencing and is performed simultaneously with the whole genome sequencing or whole exome sequencing.

### Methods

In some embodiments, the invention of the current disclosure provides a method for diagnosing and treating acute rejection (AR) of a cardiac transplant tissue in a subject, the method comprising contacting a biological sample from the subject with a reagent for assaying the level of at least one biomarker signature nucleic acid, detecting the amount of the at least one biomarker signature nucleic acid in the biological sample using the reagent, comparing the level of the at least one biomarker signature nucleic acid from the biological sample to the level of the at least one biomarker signature nucleic acid from a control sample, determining whether the level of the at least one biomarker signature nucleic acid in the biological sample is an equivalent level, an increased level or a decreased level of the at least one biomarker signature nucleic acid compared to the control sample, wherein an increased level or a decreased level of the at least one biomarker signature nucleic acid in the biological sample relative to the level of the at least one biomarker signature nucleic acid from the control sample indicates that the subject has or is at risk of acute rejection of the cardiac transplant tissue, and when the subject is determined to have or be at risk of acute rejection of cardiac transplant tissue, recommending or providing a treatment to the subject. Treatments and treatment regimens used to treat transplant rejection, including acute rejection, include but are not limited to increasing the dose or frequency of rejection-ameliorating medications, changing to a different rejection medication, and adding other medications that suppress the immune system, including prednisone or similar steroid. Medications commonly used alone or in combination to treat transplant rejection can include, but are not limited to tacrolimus, cyclosporine, prednisone, mycophenolate, azathioprine, sirolimus, and everolimus among others. In certain embodiments, the biomarker signatures and methods of the invention may be used to risk-stratify subjects for more frequent clinical screening. The skilled artisan would be able to select a new treatment regimen or alter an existing regimen using the biomarker signatures of the invention. Furthermore, individuals may be risk-stratified for more frequent clinical screening, which may include, but is not limited to, cell-free DNA assessment, to reduce the likelihood of rejection episodes. In certain embodiments, the at least one biomarker signature nucleic acid is a gene product (e.g., RNA) from a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, ZCCHC12, and any combination thereof. In certain preferred embodiments, the biological signature gene product nucleic acid is RNA. It is contemplated that any RNA molecule generated by transcription of a signature gene could be used as a biomarker in the invention, including but not limited to pre-mRNA, mRNA, micro-RNA, non-coding RNA, ribosomal RNA, transfer RNA, small nuclear RNA, small nucleolar RNA, and the like. In certain embodiments, the detecting of the at least one biomarker signature nucleic acid in the biological sample is accomplished by RNA-seq. In certain embodiments, the biological sample is an endomyocardial biopsy. In certain embodiments, the biological sample is peripheral blood.

In another aspect, the invention of the current disclosure provides a method for determining whether a subject is at risk of developing acute rejection (AR) of a cardiac transplant tissue and then treating the subject therefor, the method comprising contacting a biological sample from the subject with a reagent for assaying the level of at least one biomarker signature nucleic acid product (e.g., RNA) of a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, and ZCCHC12, detecting the amount of the at least one biomarker signature nucleic acid in the biological sample using the reagent, comparing the level of the at least one biomarker signature nucleic acid from the biological sample to the level of the at least one biomarker signature nucleic acid from a control sample, and determining whether the level of the at least one biomarker signature nucleic acid in the biological sample is an equivalent level, an increased level or a decreased level of the at least one biomarker signature nucleic acid compared to the control sample, wherein an increased level or a decreased level of the at least one signature nucleic acid in the biological sample relative to the level of the at least one biomarker signature nucleic acid from the control sample indicates that the subject is at risk of developing acute rejection, and when the subject is at risk of developing acute rejection of a cardiac transplant, recommending or providing a treatment to the subject. Treatments and treatment regimens used to treat transplant rejection, including acute rejection, include but are not limited to increasing the dose or frequency of rejection-ameliorating medications, changing to a different rejection medication, and adding other medications that suppress the immune system, including prednisone or similar steroid. Medications commonly used alone or in combination to treat transplant rejection can include, but are not limited to tacrolimus, cyclosporine, prednisone, mycophenolate, azathioprine, sirolimus, and everolimus among others. In certain embodiments, the biomarker signatures and methods of the invention may be used to risk-stratify subjects for more frequent clinical screening. The skilled artisan would be able to select a new treatment regimen or alter an existing regimen using the biomarker signatures of the invention. In certain preferred embodiments, the biological signature gene product nucleic acid is RNA. It is contemplated that any RNA molecule generated by transcription of a signature gene could be used as a biomarker in the invention, including but not limited to pre-mRNA, mRNA, micro-RNA, non-coding RNA, ribosomal RNA, transfer RNA, small nuclear RNA, small nucleolar RNA, and the like. In certain embodiments, the detecting of the at least one biomarker signature nucleic acid in the biological sample is accomplished by RNA-seq. In certain embodiments, the biological sample is an endomyocardial biopsy. In certain embodiments, the biological sample is peripheral blood.

### Kits

The invention provides kits for the detection of biomarker signature gene products, which are indicative of the presence of one or more biological sequences (e.g. RNA sequences) associated with transplant rejection. The kits may be used for detecting the expression levels of multiple gene products associated with transplant rejection. The kits may be used for the diagnosis or detection of different stages of transplant rejection, including acute rejection.

In certain embodiments, the kit or assay device of the invention comprises a composition comprising at least two biomarker signature gene products useful for diagnosing, predicting, and/or monitoring transplant rejection in a sample of a subject, wherein the at least two biomarker signature proteins are selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, and ZCCHC12, or fragments, or variants thereof.

In certain embodiments, the kit comprises one or more sterile containers which contain reagents sufficient to measure the expression of a biomarker signature gene products. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The instructions will generally include information about the use of the composition for the detection or diagnosis of transplant rejection. In certain embodiments, the instructions include information about how to use the kit. In certain embodiments the instructions include information on how to analyze and interpret the data generated from use of the kit. In other embodiments, the instructions include at least one of the following: description of the therapeutic agent; dosage schedule and administration for treatment or prevention of transplant rejection or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Sample Preparation

The invention provides a means for analyzing the levels or concentrations of multiple types of nucleic acids and/or polypeptides present in a sample. In certain embodiments, sample preparation involves extracting a mixture of nucleic acids. In other embodiments, sample preparation involves extracting a mixture of nucleic acids from multiple organisms or cell types, or any combination thereof. In certain embodiments, sample preparation involves the collection of a endomyocardial biopsy sample from a subject, extraction of RNA from the biopsy sample, preparing whole transcriptome libraries from the RNA, and identification of nucleic acids by RNA-seq or other suitable next generation sequencing (NGS) technology.

In certain embodiments, the biological sample comprises a tissue biopsy including an endomyocardial biopsy, a blood sample, a serum sample, a plasma sample, a lymph sample, a urine sample, a saliva sample, a tear sample, a sweat sample, a semen sample, a vaginal sample, a branchial sample, a mucosal sample, a cerebrospinal fluid (CSF) sample, or brain microdialysate. In other embodiments, the level of said at least one signature gene is measured or detected using an immunoassay, a western blot analysis, mass spectrometry, tandem mass (MS/MS) spectrometry, multiplexed tandem mass-tag, liquid chromatography (LC) fractionation, TOMAHAQ, a TMT-LC/LC/MS/MS platform, a next-generation sequencing platform, an RNA-seq platform, or an ultra-deep proteomic platform. In another embodiment, the samples comprises a reagent that is useful for performing an immunoassay, a western blot analysis, a mass spectrometry analysis, a tandem mass (MS/MS) spectrometry analysis, a multiplexed tandem mass-tag analysis, a liquid chromatography (LC) fractionation analysis, a TOMAHAQ analysis, a TMT-LC/LC/MS/MS platform analysis, a next-generation sequencing analysis, an RNA-seq analysis, or an ultra-deep proteomic platform analysis. In certain embodiments, the reagent comprises nucleic acid sequences, nucleic acid-specific sequences, and wherein the detecting or measuring comprises next-generation sequencing.

### Diagnostic Assays

The present invention provides a number of diagnostic assays that are useful for the identification or characterization of a disease or disorder (e.g., transplant rejection), or a propensity to develop such a condition, or a risk of developing a more severe stage of the condition. In one embodiment, transplant rejection is characterized by quantifying the level of one or more biomarker gene products whose expression is associated with the development or progression of transplant rejection. While the examples provided herein describe specific methods of detecting levels of these biomarkers, the skilled artisan appreciates that the invention is not limited to such methods. Marker levels are quantifiable by any standard method, such methods include, but are not limited to real-time PCR, Southern blot, PCR, RNA-seq, next-generation sequencing, microarrays, and/or mass spectroscopy.

The level of any two or more of the biomarkers described herein defines the biomarker profile of a disease, disorder, or condition. The level of biomarker is compared to a reference. In one embodiment, the reference is the level of marker present in a control sample obtained from a patient that is not undergoing transplant rejection. In another embodiment, the reference is a healthy tissue or cell (i.e., that is negative for rejection). In another embodiment, the reference is a baseline level of marker present in a biologic sample derived from a patient prior to, during, or after a cardiac transplant. In yet another embodiment, the reference is a standardized curve. The level of any one or more of the markers described herein is used, alone or in combination with other standard methods, to characterize the disease, disorder, or condition (e.g., transplant rejection).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed herein.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the exemplary embodiments of the present invention and are not to be construed as limiting in any way the remainder of the disclosure.

The materials and methods used in the following experimental examples are now provided.

Clinical Trial of Transplantation (CTOT-03) Study: The CTOT-03 study is described in detail elsewhere (NCT:00531921) but in brief it is a prospective observational cohort study designed to test associations of proinflammatory pathways of allo-immune response and injury in thoracic (heart and lung) and abdominal (kidney and liver) allografts. The study aims include testing associations between mRNA expression and subsequent incidence of acute rejection and expression of genes involved in cell mediated immunity in recipients of kidney, liver, lung and heart transplants. For the purposes of this study, RNA-seq data were generated from 125 EMBs from 26 CTOT-03 patients recruited from the University of Pennsylvania and the University of Wisconsin, to assess effects of heart allograft gene expression and the relationship with acute rejection.

A dedicated research EMB was collected, at the same time, for up to six standard-of-care and/or for-cause timepoints in the first 12-months post-transplant, and these fresh-frozen EMBs were preserved immediately in RNA later storage buffer (Thermo Fisher) at −80° C. A representative hematoxylin-eosin (H&E)-stained slide for each clinical biopsy was centralized in each of the two CTOT-03 sites and graded using International Society of Heart and Lung Transplantation consensus definitions. A dedicated Fresh-frozen research EMBs was collected from the donor allograft at the day of transplant (Day 0) and at one week, two weeks, one month, three months, six months and one-year post-transplant. For-cause research fresh-frozen EMB timepoints were also collected. A dedicated Fresh-frozen research EMBs was collected from the donor allograft at the day of transplant (Day 0) and at one week, two weeks, one month, three months, six months and one-year post-transplant. For-cause research fresh-frozen EMB timepoints were also collected. The EMBs were further independently assessed by two blinded pathologists to arrive at a consensus rejection status grade with adjudication where required. The distribution of grades across the sample cohort is shown in Table 1.

Tissue extraction: Flash-frozen EMB tissues in RNAlater buffer were homogenized by rotor-stator homogenizer (TissueRuptor, Qiagen), and RNA was purified from the homogenized lysate using RNeasy Blood and Tissue kits (Qiagen). RNA quantity and quality were assessed on a BioAnalyzer workstation (Agilent) and by Qubit fluorometer (Thermo Fisher). Whole transcriptome libraries were prepared using TruSeq Stranded Total RNA Gold library preparation kits (Illumina) and were multiplexed for RNA sequencing on a HiSeq 2500 instrument (Illumina).

Data analysis: For RNA-seq libraries, raw data were demultiplexed and converted to FASTQ using bcl2fastq2 (Illumina). Reads were mapped and quantified to the Ensembl hg38 human reference genome build using the Salmon. Salmon quants were read into R and rolled up to gene level using the tximport package. Initial quality control checks determined 9 samples failed sequencing due to low read counts and were removed from downstream analyses. The raw feature counts for the remaining 116 samples were normalized using the edgeR package in R and differentially expressed genes were calculated using the exact test function in edgeR with false-discovery rate correction (q<0.01). Pathway analysis was performed using DAVID pathway analysis tools 22, 23, utilizing biological pathways from the Kyoto Encyclopedia of Genes and Genomes (KEGG) and Gene Ontology (GO) Immune cell deconvolution was conducted on the contrasts of interest via xCell. Predictor models for classification of rejection and preceding rejection contrasts were constructed using the randomForest package in R. Random forest is a machine learning approach for classification of covariates or biomarkers that optimizes across a large number of decision trees, and is particularly useful for classification across high-dimensional datasets such as RNA-seq. The random forest models were subjected to 10-fold cross validation in which each respective model was trained on 55% of the samples and tested for classification of rejection grade or preceding-rejection on the remaining 45% of samples which were sampled without replacement. Due to vastly more non-rejection samples in the cohort, for the random forest training and test sets the non-rejection samples were downsampled without replacement to twice the number of rejection samples. Similarly, for the preceding-rejection classifier the preceding-non-rejection samples were down-sampled to twice the number of preceding-rejection samples. The random forest classifier models were evaluated after extracting the true positive, true negative, false positive, and false negative values for each round of cross validation. The models were assessed using metrics of accuracy, sensitivity, specificity, positive predictive value (PPV), negative predictive value (NPV), F1 score, false positive rate (FPR), false negative rate (FNR), false discovery rate (FDR), and Matthews correlation coefficient (MCC). MCC is a metric ranging from −1 to 1 to evaluate the quality of classification with 1 representing a perfect classification with agreement of actual and predicted values in all four categories of the confusion matrix and −1 representing a very poor classification with a high rate of disagreement between actual and predicted values that could be generated by random. Accuracy of classification was calculated on the test set by obtaining the fraction for the number of correct predictions out of the total number of samples in the test set. Accuracy of the rejection grade and preceding rejection signatures respectively were assessed through ten rounds of cross-validation with resampling of the training and testing sets each round. An average for each metric was calculated from the ten rounds of cross validation for each respective random forest model. Area under the curve (AUC) metrics and receiver-operating characteristic (ROC) curve plots were generated using the ROCR package in R.

TABLE 1
Demographics and Clincal Features of
CTOT03 Heart Transplant Participants

| | |
|---|---|
| Patients | Recipients |
| without ACR | with 2R EMBs |

| | | |
|---|---|---|
| Number of patients | 19 | 7 |
| Donors | | |

| Age (Median[IQR]) | 37 | (21-52) | 21 | (16-32) |
|---|---|---|---|---|
| Gender (Male n, %) | 16 | (84%) | 6 | (85%) |
| Race | | | | |
| African American (n, %) | 2 | (11%) | 0 | (0%) |
| White (n, %) | 16 | (84%) | 5 | (71%) |
| Unknown (n, %) | 1 | (5%) | 2 | (29%) |
| Recipients | | | | |
| Age (Median[IQR]) | 60 | (53-61) | 56 | (46-58) |
| Gender (Male n, %) | 16 | (84%) | 5 | (71%) |
| Indications for Heart Transplant | | | | |
| Idiopathic | 2 | (11%) | 3 | (43%) |
| Ischemic | 13 | (68%) | 2 | (29%) |
| Other | 4 | (21%) | 2 | (29%) |
| Ancestry | | | | |
| African American (n, %) | 2 | (11%) | 1 | (14%) |
| White (n, %) | 16 | (84%) | 5 | (71%) |
| Unknown (n, %) | 1 | (5%) | 1 | (14%) |
| Study site (HUP, n, %) | 12 | (63%) | 3 | (43%) |

| | | | |
|---|---|---|---|
| Time to 1st ACR (≥2R) | NA | 58 | [9-165] |
| (Median [IQR]) | | | |

### Example 1: A Gene Expression Signature is Associated with Acute Rejection in Endomyocardial Biopsies (EMBs)

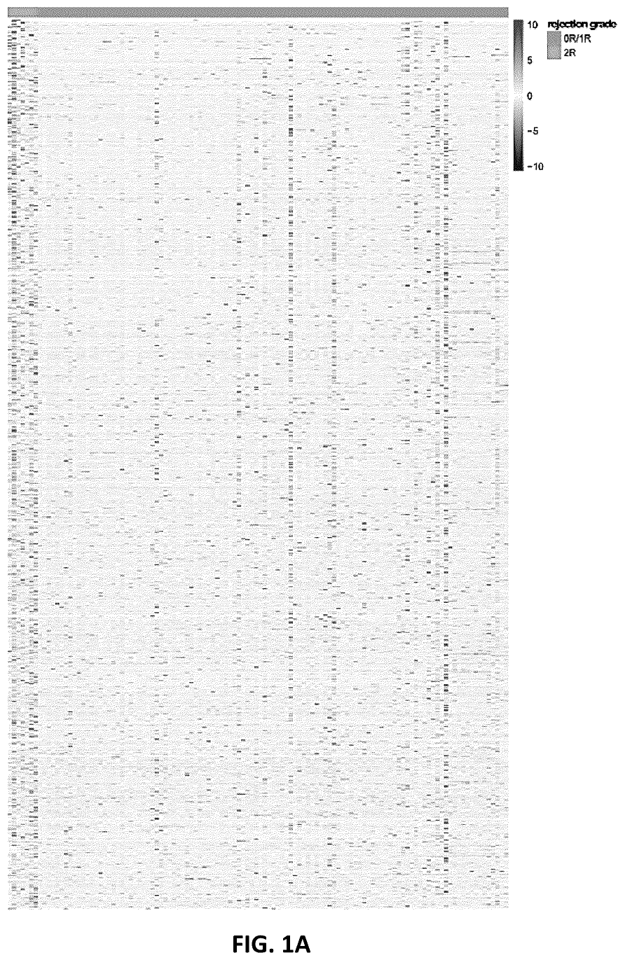

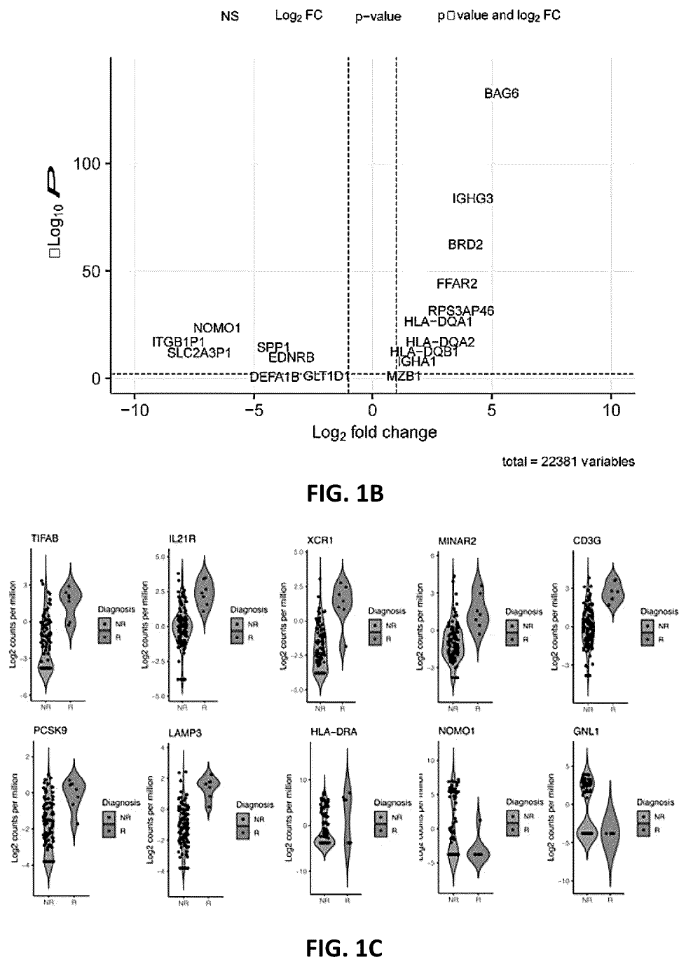

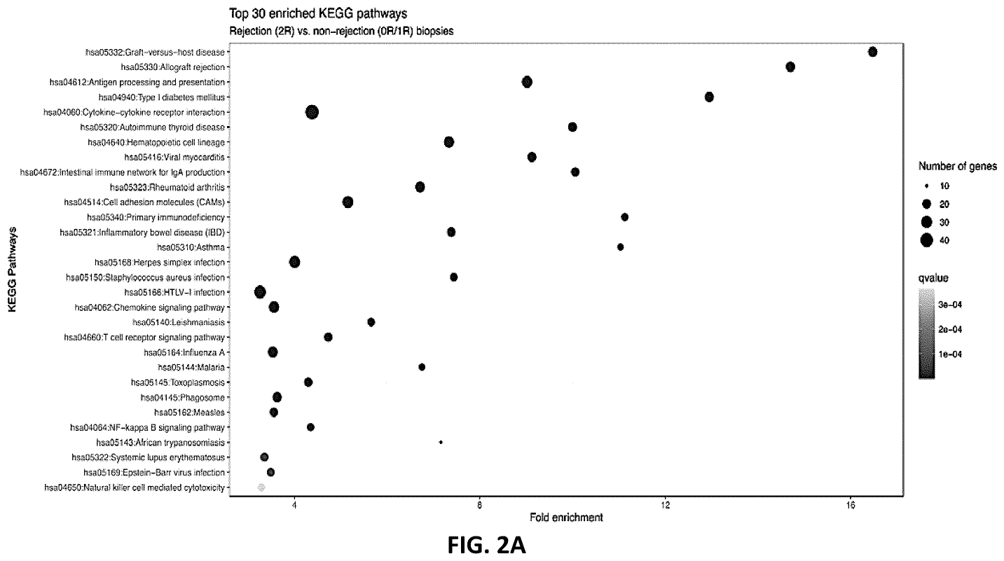

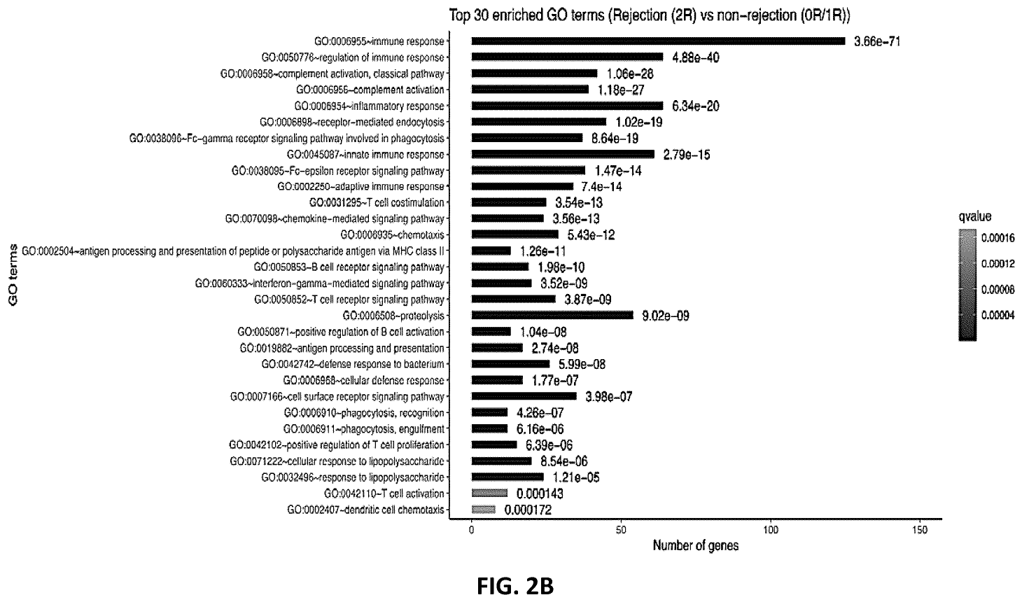

Longitudinal fresh-frozen EMBs were available from 50 heart transplant recipients who were enrolled in the CTOT-03 study (see Methods). From this cohort, a subset of 26 patients were selected that had dense sampling of EMBs over the study period for allograft profiling by RNA-seq. In total 125 tissue samples were profiled by RNA-seq. Studies first assessed expression differences between histopathological-determined rejection (Grade 2R or 3R) and non-rejection states (Grade 0R or 1R) as defined by ISHLT grading. Across the 26 CTOT-03 heart transplant recipients, there were 59 EMBs graded as 0R, 59 graded as 1R and seven EMBs were graded as 2R rejection episodes. A transcriptome-wide Fisher's exact test and pathway analyses was performed in order to assess differences between 0R/1R and 2R samples. From these data, 1079 genes were observed to be significantly different between 0R/1R and 2R (FDR q<0.01, Benjamini-Hochberg correction) (FIGS. 1A & 1B and Table 2). Of note, a number of these differentially expressed genes have previously been linked to transplant-associated phenotypes, including elevated PCSK9 association with immunosuppressive therapy and HLA-DRA associations in peripheral blood with AR 26 (FIG. 1C). Genes downregulated in the AR setting included NOMO1, which is involved in inhibiting cardiomyocyte differentiation, FOSB which is a known respond molecule to cardiac injury, and EDNRB, a gene which is associated with cardiac stress tolerance. To further elucidate functional pathways associated with AR, pathway analyses was performed using the DAVID tool, which assesses whether a differentially expressed gene list is statistically enriched for predefined sets of genes based on biological function or other properties (FIGS. 2A and 2B). The majority of significant pathways between 0R/1R and 2R/3R biopsies were associated with immune responses including KEGG cytokine-cytokine receptor interaction (q<1.54×10-15) and a number of pathways significantly associated with transplant rejection including the KEGG Graft-Versus-Host Disease pathway (q<6.37×10⁻²¹) and KEGG Allograft Rejection (q<1.31×10⁻¹⁹). For the latter pathway, significant genes comprised most facets of AR including donor and recipient antigen presentation, T helper cell mediated immune activation and both cytotoxic CD8⁺ T cells (CTL) and macrophage derived donor tissue destruction. As the majority of significant differences between healthy and AR EMBs were associated with immune-related pathways, it was next assessed which classes of immune cells were activated in AR tissues. Immune cell deconvolution of the RNA-seq dataset using the xCell algorithm was performed, which estimates the abundance of specific immune cell subtypes in a sample based on the RNA expression levels of cell-specific markers. Consistent with pathway analysis, AR EMBs were enriched for CD8+ naive T-cells (q<0.05).

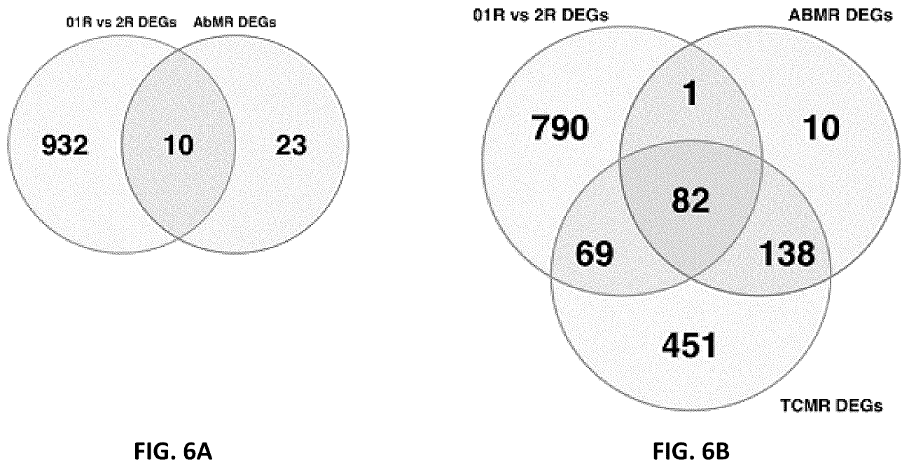

Studies then assessed overlap of the AR signature with other previously identified signatures. This includes the antibody mediated rejection (AbMR) signature previously identified in Loupy et al. as well as T-cell mediated rejection (TCMR) and AbMR signatures curated from large datasets by Xiu et al. For the former, it was observed that ˜30% of genes in the Loupy et al. signature, as well as ˜20% of the genes in the TCMR signature, are present in our AR signature (FIG. 6A). Furthermore, ˜36% of the genes in the AbMR signature from Xiu et al. (FIG. 6B) are present in our AR signature, indicating that while a subset of the characterized genes are novel, there is concordance with prior studies in this area.

TABLE 2
List of significant genes for the 0R/1R vs 2R/3R comparison.

| Ensmbl_ID | logFC | logCPM | PValue | FDR | Gene_symbol |
|---|---|---|---|---|---|
| ENSG00000227761 | 5.41 | −0.06 | 5.80E−138 | 1.30E−133 | BAG6 |
| ENSG00000211897 | 4.21 | 0.79 | 6.08E−89 | 6.81E−85 | IGHG3 |
| ENSG00000234507 | 3.90 | 0.51 | 1.41E−67 | 1.05E−63 | BRD2 |
| ENSG00000126262 | 3.55 | 1.77 | 2.18E−49 | 1.22E−45 | FFAR2 |
| ENSG00000226132 | 3.72 | −0.89 | 1.06E−36 | 4.76E−33 | RPS3AP46 |
| ENSG00000243290 | 3.05 | −0.62 | 6.62E−36 | 2.47E−32 | IGKV1-12 |
| ENSG00000235655 | −10.00 | 3.13 | 8.84E−34 | 2.83E−30 | H3P6 |
| ENSG00000206305 | 2.77 | 3.43 | 1.01E−31 | 2.82E−28 | HLA-DQA1 |
| ENSG00000231939 | 2.75 | 1.31 | 2.19E−30 | 5.45E−27 | HLA-DQB1 |
| ENSG00000173578 | 2.88 | −0.43 | 2.33E−29 | 5.21E−26 | XCR1 |
| ENSG00000242221 | 3.21 | −0.36 | 3.83E−29 | 7.80E−26 | PSG2 |
| ENSG00000103512 | −6.53 | 4.33 | 1.10E−28 | 2.06E−25 | NOMO1 |
| ENSG00000213269 | 3.72 | −1.60 | 1.62E−28 | 2.80E−25 | RPS6P26 |
| ENSG00000211955 | 2.71 | −0.09 | 3.64E−28 | 5.82E−25 | IGHV3-33 |
| ENSG00000211961 | 3.46 | −2.25 | 2.51E−27 | 3.60E−24 | IGHV1-45 |
| ENSG00000244116 | 2.62 | 0.15 | 2.57E−27 | 3.60E−24 | IGKV2-28 |
| ENSG00000225840 | −5.25 | 17.72 | 7.42E−27 | 9.78E−24 | |
| ENSG00000213886 | 2.63 | 0.22 | 9.75E−27 | 1.21E−23 | UBD |
| ENSG00000226060 | 3.87 | −2.39 | 7.61E−26 | 8.97E−23 | TRIM26 |
| ENSG00000243264 | 2.96 | −1.64 | 8.12E−26 | 9.09E−23 | IGKV2D-29 |
| ENSG00000101489 | 2.67 | 1.82 | 9.52E−25 | 1.01E−21 | CELF4 |
| ENSG00000215048 | 2.54 | −0.43 | 1.08E−24 | 1.10E−21 | HLA-DPB1 |
| ENSG00000206492 | −8.48 | 1.64 | 5.50E−24 | 5.36E−21 | GNL1 |
| ENSG00000253497 | 3.89 | −2.52 | 9.79E−23 | 9.14E−20 | IGKV1-13 |
| ENSG00000174460 | 2.99 | −1.79 | 1.15E−22 | 1.03E−19 | ZCCHC12 |
| ENSG00000105205 | 2.73 | −0.58 | 1.49E−22 | 1.28E−19 | CLC |
| ENSG00000269378 | −8.15 | 1.33 | 3.50E−22 | 2.90E−19 | ITGB1P1 |
| ENSG00000223793 | 2.85 | −1.86 | 3.75E−22 | 3.00E−19 | HLA-DQA2 |
| ENSG00000149516 | 3.08 | −1.81 | 4.70E−22 | 3.63E−19 | MS4A3 |
| ENSG00000117215 | 2.65 | −0.91 | 3.61E−21 | 2.69E−18 | PLA2G2D |
| ENSG00000211673 | 2.36 | −0.17 | 4.10E−21 | 2.96E−18 | IGLV3-1 |
| ENSG00000242386 | 2.54 | −0.63 | 6.08E−21 | 4.26E−18 | HLA-DMB |
| ENSG00000243238 | 2.41 | −0.56 | 8.29E−21 | 5.63E−18 | IGKV2-30 |
| ENSG00000206356 | 3.00 | −0.77 | 9.02E−21 | 5.94E−18 | |
| ENSG00000211935 | 2.57 | −1.29 | 1.50E−20 | 9.59E−18 | IGHV1-3 |
| ENSG00000235715 | 2.31 | 0.88 | 3.39E−20 | 2.11E−17 | PSMB8 |
| ENSG00000206468 | 2.29 | 0.92 | 4.81E−20 | 2.91E−17 | UBD |
| ENSG00000240107 | 3.24 | −1.64 | 1.14E−19 | 6.72E−17 | |
| ENSG00000118785 | −4.17 | 5.35 | 1.31E−19 | 7.51E−17 | SPP1 |
| ENSG00000197753 | 3.26 | −2.02 | 1.48E−19 | 8.31E−17 | LHFPL5 |
| ENSG00000149742 | 3.01 | −1.16 | 1.58E−19 | 8.65E−17 | SLC22A9 |
| ENSG00000211941 | 2.59 | −1.53 | 1.82E−19 | 9.50E−17 | IGHV3-11 |
| ENSG00000204619 | 2.34 | −0.19 | 1.82E−19 | 9.50E−17 | PPP1R11 |
| ENSG00000125740 | −4.20 | 3.42 | 3.82E−19 | 1.94E−16 | FOSB |
| ENSG00000232196 | 2.22 | 8.97 | 5.63E−19 | 2.80E−16 | MTRNR2L4 |
| ENSG00000211662 | 2.25 | −0.23 | 9.03E−19 | 4.40E−16 | IGLV3-21 |
| ENSG00000144771 | 2.51 | 0.40 | 1.73E−18 | 8.25E−16 | LRTM1 |
| ENSG00000144820 | −6.59 | 0.75 | 2.18E−18 | 1.02E−15 | ADGRG7 |
| ENSG00000255833 | 2.27 | 0.11 | 2.22E−18 | 1.02E−15 | TIFAB |
| ENSG00000204287 | 2.18 | 3.57 | 2.59E−18 | 1.16E−15 | HLA-DRA |
| ENSG00000206292 | 2.22 | 0.53 | 2.93E−18 | 1.29E−15 | HLA-DOA |
| ENSG00000236271 | −4.91 | 0.98 | 3.83E−18 | 1.65E−15 | FLOT1 |
| ENSG00000211669 | 2.52 | −1.74 | 4.06E−18 | 1.71E−15 | IGLV3-10 |
| ENSG00000251039 | 3.49 | −2.54 | 4.12E−18 | 1.71E−15 | IGKV2D-40 |
| ENSG00000198576 | −4.75 | 1.26 | 6.29E−18 | 2.56E−15 | ARC |
| ENSG00000179242 | 3.10 | −1.43 | 8.10E−18 | 3.24E−15 | CDH4 |
| ENSG00000185046 | 2.23 | 2.52 | 9.92E−18 | 3.90E−15 | ANKS1B |
| ENSG00000206302 | 2.19 | 1.16 | 1.04E−17 | 4.02E−15 | HLA-DQB1 |
| ENSG00000231286 | 2.18 | 2.32 | 1.34E−17 | 5.07E−15 | HLA-DQB1 |
| ENSG00000225698 | 2.75 | −1.96 | 2.76E−17 | 1.03E−14 | IGHV3-72 |
| ENSG00000249439 | 2.87 | −1.43 | 2.87E−17 | 1.05E−14 | HMGN1P14 |
| ENSG00000253861 | −7.27 | 0.51 | 4.22E−17 | 1.52E−14 | SLC2A3P1 |
| ENSG00000069011 | 2.62 | −0.80 | 4.69E−17 | 1.67E−14 | PITX1 |
| ENSG00000163631 | −5.60 | 0.73 | 5.17E−17 | 1.81E−14 | ALB |
| ENSG00000241386 | 2.53 | −1.64 | 7.03E−17 | 2.41E−14 | HLA-DOB |
| ENSG00000251705 | −3.69 | 5.80 | 7.09E−17 | 2.41E−14 | RNA5-8SP6 |
| ENSG00000172724 | 2.11 | 1.46 | 7.37E−17 | 2.46E−14 | CCL19 |
| ENSG00000211967 | 2.57 | −1.91 | 1.22E−16 | 4.01E−14 | IGHV3-53 |
| ENSG00000160654 | 2.19 | 1.02 | 2.03E−16 | 6.57E−14 | CD3G |
| ENSG00000211892 | 2.14 | −0.30 | 2.16E−16 | 6.92E−14 | IGHG4 |
| ENSG00000241132 | 2.46 | −1.42 | 3.76E−16 | 1.19E−13 | LY6G5B |
| ENSG00000241674 | 2.33 | −0.77 | 3.92E−16 | 1.22E−13 | HLA-DMB |
| ENSG00000239618 | 2.45 | −1.04 | 4.89E−16 | 1.50E−13 | HLA-L |
| ENSG00000227766 | 3.15 | −2.40 | 5.85E−16 | 1.77E−13 | |
| ENSG00000231475 | 2.94 | −2.34 | 6.15E−16 | 1.84E−13 | IGHV4-31 |
| ENSG00000244207 | −5.84 | 0.78 | 1.34E−15 | 3.94E−13 | C4A |
| ENSG00000228927 | 3.12 | −2.30 | 2.71E−15 | 7.87E−13 | TSPY3 |
| ENSG00000206297 | 2.76 | −1.97 | 2.89E−15 | 8.31E−13 | TAP1 |
| ENSG00000188257 | −3.47 | 3.63 | 3.32E−15 | 9.41E−13 | PLA2G2A |
| ENSG00000231558 | 2.07 | 1.10 | 3.53E−15 | 9.88E−13 | HLA-DOA |
| ENSG00000186367 | 2.28 | 0.21 | 3.62E−15 | 1.00E−12 | MINAR2 |
| ENSG00000136160 | −3.42 | 3.83 | 4.11E−15 | 1.12E−12 | EDNRB |
| ENSG00000184611 | 3.61 | −2.57 | 4.18E−15 | 1.13E−12 | KCNH7 |
| ENSG00000211952 | 2.79 | −2.25 | 4.28E−15 | 1.14E−12 | IGHV4-28 |
| ENSG00000169429 | −3.66 | 2.43 | 4.46E−15 | 1.18E−12 | CXCL8 |
| ENSG00000078081 | 2.11 | −0.10 | 5.39E−15 | 1.40E−12 | LAMP3 |
| ENSG00000166104 | 2.79 | −2.14 | 5.56E−15 | 1.43E−12 | |
| ENSG00000224041 | 2.13 | −1.27 | 1.46E−14 | 3.73E−12 | IGKV3D-15 |
| ENSG00000211950 | 2.57 | −2.00 | 1.85E−14 | 4.65E−12 | IGHV1-24 |
| ENSG00000244681 | 2.07 | 1.41 | 1.89E−14 | 4.70E−12 | MTHFD2P1 |
| ENSG00000186810 | 2.11 | −0.45 | 2.47E−14 | 6.07E−12 | CXCR3 |
| ENSG00000185352 | −3.65 | 1.20 | 2.97E−14 | 7.23E−12 | HS6ST3 |
| ENSG00000109684 | 2.14 | −0.65 | 3.13E−14 | 7.55E−12 | CLNK |
| ENSG00000241106 | 2.10 | −0.79 | 4.63E−14 | 1.10E−11 | HLA-DOB |
| ENSG00000230475 | −3.57 | 1.16 | 5.02E−14 | 1.18E−11 | ABHD16A |
| ENSG00000254395 | 2.79 | −2.33 | 5.13E−14 | 1.20E−11 | IGHV4-55 |
| ENSG00000131401 | 1.99 | 0.80 | 5.24E−14 | 1.21E−11 | NAPSB |
| ENSG00000110448 | 2.04 | 0.52 | 6.46E−14 | 1.48E−11 | CD5 |
| ENSG00000159307 | 2.22 | 1.10 | 6.78E−14 | 1.53E−11 | SCUBE1 |
| ENSG00000206503 | 1.93 | 4.49 | 6.87E−14 | 1.54E−11 | HLA-A |
| ENSG00000163132 | 2.05 | 1.70 | 7.21E−14 | 1.60E−11 | MSX1 |
| ENSG00000211934 | 2.02 | −0.73 | 8.00E−14 | 1.76E−11 | IGHV1-2 |
| ENSG00000231608 | −3.17 | 4.33 | 8.32E−14 | 1.81E−11 | TNXB |
| ENSG00000213889 | 2.05 | 1.96 | 8.78E−14 | 1.89E−11 | PPM1N |
| ENSG00000186265 | 2.07 | 0.16 | 9.87E−14 | 2.10E−11 | BTLA |
| ENSG00000138795 | 1.99 | 0.54 | 1.04E−13 | 2.19E−11 | LEF1 |
| ENSG00000137101 | 2.02 | −0.24 | 1.21E−13 | 2.54E−11 | CD72 |
| ENSG00000112486 | 2.15 | −0.87 | 1.31E−13 | 2.72E−11 | CCR6 |
| ENSG00000230143 | 2.69 | −2.12 | 1.81E−13 | 3.73E−11 | FLOT1 |
| ENSG00000179934 | 2.64 | −2.15 | 1.99E−13 | 4.04E−11 | CCR8 |
| ENSG00000242092 | 1.89 | 1.83 | 2.47E−13 | 4.98E−11 | HLA-DMB |
| ENSG00000211895 | 1.88 | 3.76 | 3.57E−13 | 7.15E−11 | IGHA1 |
| ENSG00000253120 | 3.51 | −2.70 | 4.02E−13 | 7.97E−11 | IGLV2-34 |
| ENSG00000111732 | 2.32 | −1.12 | 4.84E−13 | 9.52E−11 | AICDA |

TABLE 3
List of significant genes for the Preceding Rejection
vs Preceding Non-Rejection comparison.

| Ensembl_ID | logFC | logCPM | PValue | FDR | Symbol |
|---|---|---|---|---|---|
| ENSG00000137673 | 3.70 | 0.00 | 2.35E−47 | 5.27E−43 | MMP7 |
| ENSG00000235655 | −10.09 | 3.16 | 3.38E−36 | 3.78E−32 | H3P6 |
| ENSG00000226025 | 3.11 | −0.38 | 9.25E−36 | 6.91E−32 | LGALS17A |
| ENSG00000103512 | −6.72 | 4.43 | 2.33E−35 | 1.30E−31 | NOMO1 |
| ENSG00000240864 | 3.02 | −0.78 | 1.62E−31 | 7.24E−28 | IGKV1-16 |
| ENSG00000240382 | 3.13 | −0.98 | 5.57E−31 | 2.08E−27 | IGKV1-17 |
| ENSG00000138755 | 2.68 | 5.81 | 3.32E−29 | 1.06E−25 | CXCL9 |
| ENSG00000211943 | 2.89 | −0.73 | 2.02E−28 | 5.65E−25 | IGHV3-15 |
| ENSG00000204619 | 2.72 | −0.26 | 3.67E−26 | 9.14E−23 | PPP1R11 |
| ENSG00000260596 | 2.71 | 0.25 | 4.41E−26 | 9.87E−23 | DUX4 |
| ENSG00000269378 | −8.53 | 1.64 | 1.18E−25 | 2.41E−22 | ITGB1P1 |
| ENSG00000215048 | 2.73 | −0.76 | 1.34E−25 | 2.50E−22 | HLA-DPB1 |
| ENSG00000187288 | 2.77 | −0.51 | 2.37E−25 | 4.08E−22 | CIDEC |
| ENSG00000177807 | 4.67 | −2.51 | 5.58E−25 | 8.93E−22 | KCNJ10 |
| ENSG00000211938 | 2.68 | −0.54 | 8.75E−25 | 1.31E−21 | IGHV3-7 |
| ENSG00000211946 | 3.40 | −2.01 | 1.99E−24 | 2.79E−21 | IGHV3-20 |
| ENSG00000145107 | 2.76 | −0.70 | 2.80E−24 | 3.69E−21 | TM4SF19 |
| ENSG00000211649 | 2.98 | −1.49 | 7.12E−24 | 8.86E−21 | IGLV7-46 |
| ENSG00000211895 | 2.41 | 3.32 | 1.12E−22 | 1.32E−19 | IGHA1 |
| ENSG00000211893 | 3.46 | −1.94 | 2.01E−22 | 2.26E−19 | IGHG2 |
| ENSG00000258991 | 2.80 | −1.05 | 2.95E−22 | 3.14E−19 | DUX4L19 |
| ENSG00000235715 | 2.38 | 0.56 | 3.44E−22 | 3.50E−19 | PSMB8 |
| ENSG00000206468 | 2.40 | 0.74 | 2.70E−21 | 2.63E−18 | UBD |
| ENSG00000242386 | 2.65 | −0.87 | 1.09E−20 | 1.02E−17 | HLA-DMB |
| ENSG00000256514 | −7.70 | 0.86 | 1.45E−20 | 1.30E−17 | |
| ENSG00000236138 | 2.85 | −1.41 | 7.97E−20 | 6.87E−17 | DUX4L26 |
| ENSG00000134028 | 2.29 | 0.54 | 1.52E−19 | 1.26E−16 | ADAMDEC1 |
| ENSG00000251705 | −3.93 | 5.95 | 1.83E−19 | 1.46E−16 | RNA5-8SP6 |
| ENSG00000206503 | 2.23 | 4.28 | 2.61E−19 | 2.01E−16 | HLA-A |
| ENSG00000236560 | −7.47 | 0.65 | 3.52E−19 | 2.63E−16 | PPP1R11 |
| ENSG00000163631 | −5.39 | 0.88 | 3.70E−19 | 2.67E−16 | ALB |
| ENSG00000169248 | 2.23 | 3.27 | 4.17E−19 | 2.92E−16 | CXCL11 |
| ENSG00000236271 | −7.51 | 0.69 | 4.66E−19 | 3.16E−16 | FLOT1 |
| ENSG00000181092 | 2.24 | 2.33 | 6.98E−19 | 4.60E−16 | ADIPOQ |
| ENSG00000206452 | −4.05 | 2.30 | 6.11E−18 | 3.91E−15 | HLA-C |
| ENSG00000144820 | −6.11 | 0.86 | 7.10E−18 | 4.42E−15 | ADGRG7 |
| ENSG00000211651 | 3.41 | −2.31 | 7.46E−18 | 4.42E−15 | IGLV1-44 |
| ENSG00000230475 | −4.67 | 1.18 | 7.50E−18 | 4.42E−15 | ABHD16A |
| ENSG00000228760 | 2.21 | 0.17 | 8.28E−18 | 4.75E−15 | BAG6 |
| ENSG00000185149 | 2.81 | −1.72 | 1.14E−17 | 6.38E−15 | NPY2R |
| ENSG00000178397 | −4.38 | 1.56 | 3.94E−17 | 2.15E−14 | FAM220A |
| ENSG00000118785 | 2.11 | 5.71 | 4.42E−17 | 2.36E−14 | SPP1 |
| ENSG00000270188 | 2.12 | 5.58 | 5.06E−17 | 2.64E−14 | MTRNR2L11 |
| ENSG00000231834 | −3.78 | 2.19 | 6.47E−17 | 3.29E−14 | HLA-A |
| ENSG00000235307 | 2.19 | 1.29 | 8.93E−17 | 4.44E−14 | BRD2 |
| ENSG00000171004 | 2.12 | 0.77 | 1.50E−16 | 7.29E−14 | HS6ST2 |
| ENSG00000198576 | −3.70 | 1.84 | 2.76E−16 | 1.32E−13 | ARC |
| ENSG00000125740 | −3.54 | 3.42 | 3.88E−16 | 1.81E−13 | FOSB |
| ENSG00000256618 | 2.04 | 10.93 | 5.19E−16 | 2.37E−13 | MTRNR2L1 |
| ENSG00000213886 | 2.10 | 0.02 | 6.62E−16 | 2.96E−13 | UBD |
| ENSG00000242574 | 2.26 | −0.48 | 8.04E−16 | 3.53E−13 | HLA-DMB |
| ENSG00000211961 | 3.33 | −2.47 | 1.07E−15 | 4.60E−13 | IGHV1-45 |
| ENSG00000183791 | 2.73 | −1.71 | 1.40E−15 | 5.92E−13 | NA |
| ENSG00000180581 | −3.84 | 2.24 | 3.52E−15 | 1.46E−12 | SRP9P1 |
| ENSG00000225840 | −3.21 | 17.76 | 6.50E−15 | 2.64E−12 | |
| ENSG00000204287 | 1.97 | 3.37 | 6.61E−15 | 2.64E−12 | HLA-DRA |
| ENSG00000211780 | 3.07 | −2.37 | 8.50E−15 | 3.34E−12 | TRAV6 |
| ENSG00000131203 | 1.95 | 3.08 | 3.31E−14 | 1.28E−11 | IDO1 |
| ENSG00000211666 | 1.94 | 1.03 | 4.13E−14 | 1.57E−11 | IGLV2-14 |
| ENSG00000231618 | 3.83 | −2.53 | 8.01E−14 | 2.99E−11 | PPT2 |
| ENSG00000197753 | 3.20 | −2.35 | 1.03E−13 | 3.72E−11 | LHFPL5 |
| ENSG00000077152 | −4.32 | 0.25 | 1.03E−13 | 3.72E−11 | UBE2T |
| ENSG00000255823 | 1.89 | 13.03 | 1.22E−13 | 4.26E−11 | MTRNR2L8 |
| ENSG00000259029 | 2.88 | −2.18 | 1.22E−13 | 4.26E−11 | DUX4L18 |
| ENSG00000174145 | 2.97 | −2.21 | 1.44E−13 | 4.97E−11 | NWD2 |
| ENSG00000176194 | 2.14 | −0.64 | 1.84E−13 | 6.23E−11 | CIDEA |
| ENSG00000272670 | −6.35 | −0.33 | 2.57E−13 | 8.59E−11 | DOC2B |

### Example 2: A Gene Expression Classifier for 0R/1R Vs 2R Acute Rejection

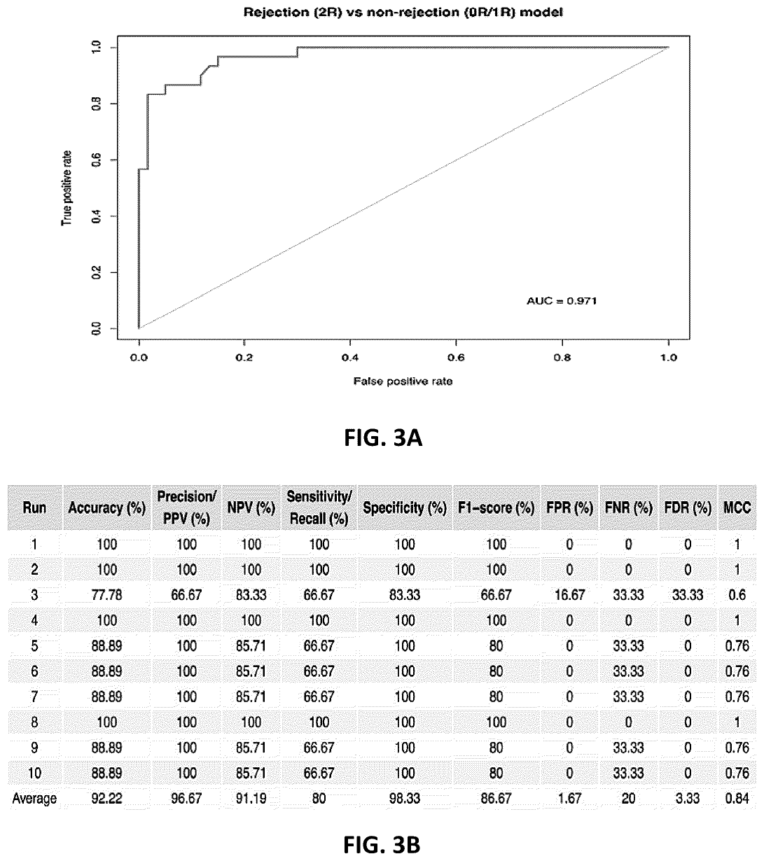

As gene expression profiling revealed robust differences between AR and non-rejection EMBs, it was next asked whether the differential gene expression signature could be used to classify samples as molecular AR or non-rejection. To do this, a predictor model was constructed using the Random Forest method to classify AR grade from the differentially expressed genes between 0R/1R and 2R/3R samples. The AR predictor model was trained on 55% of the samples, sampled with replacement and tested for classification of rejection grade on the remaining 45% of samples. Overall performance assessed using the area under the curve (AUC) of 0.971 and was plotted using a receiver-operating characteristic (ROC) curve (FIGS. 3A&B), which illustrates the AR signature's robust performance as a classifier. Classification accuracy averages 92.2%, with a positive predictive value (PPV) of 96.7%, a negative predictive value of 91.2% and a sensitivity, and specificity of 80% and 98.33%, respectively. While it will be important to evaluate this classifier across additional independent datasets, these results suggest that RNA-seq is a highly accurate methodology for classifying rejection and non-rejection specimens.

### Example 3: A Prognostic Signature for Acute Rejection

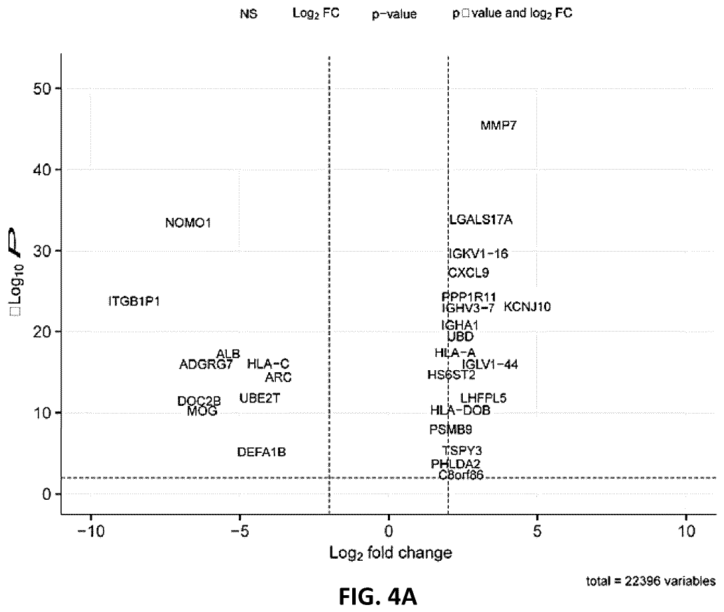

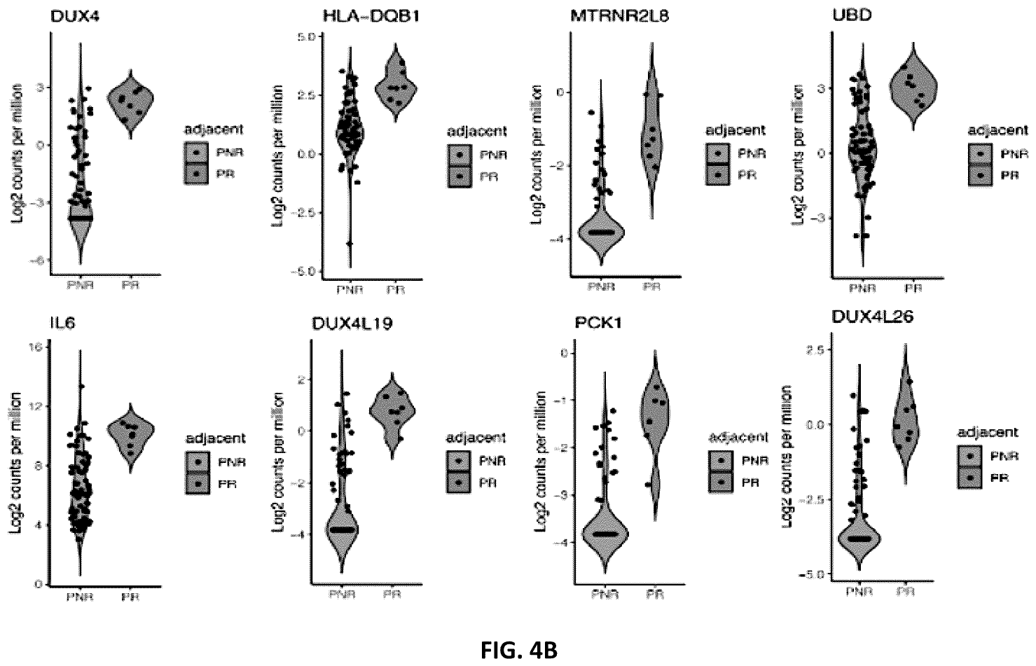

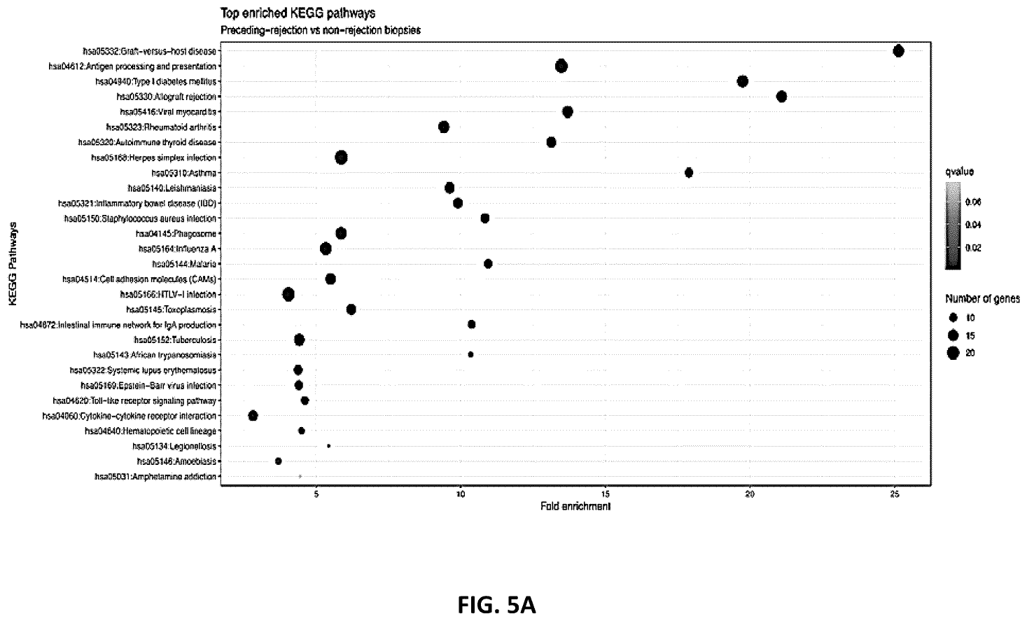

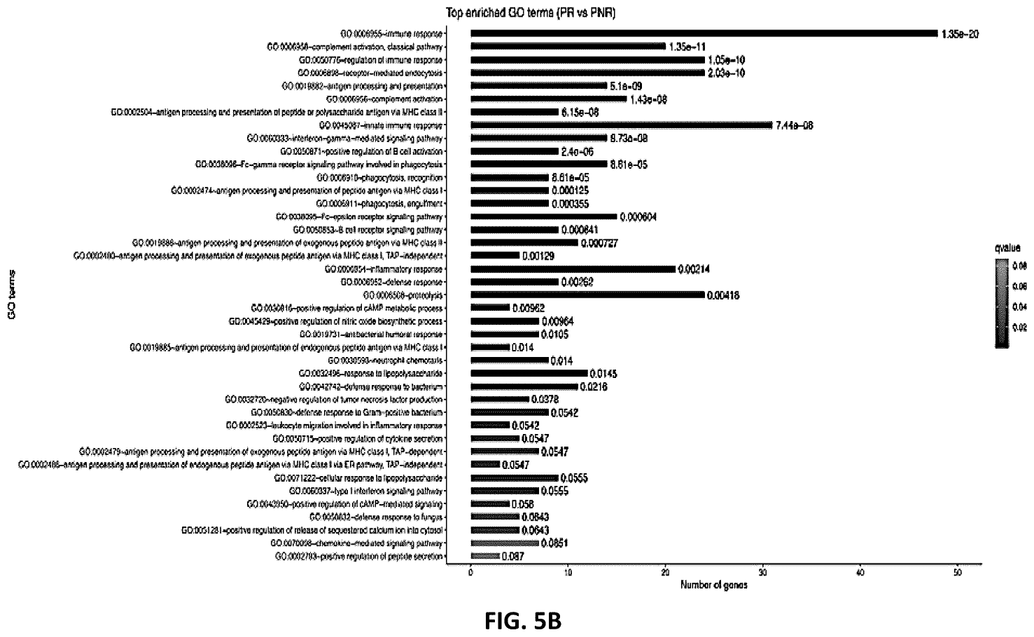

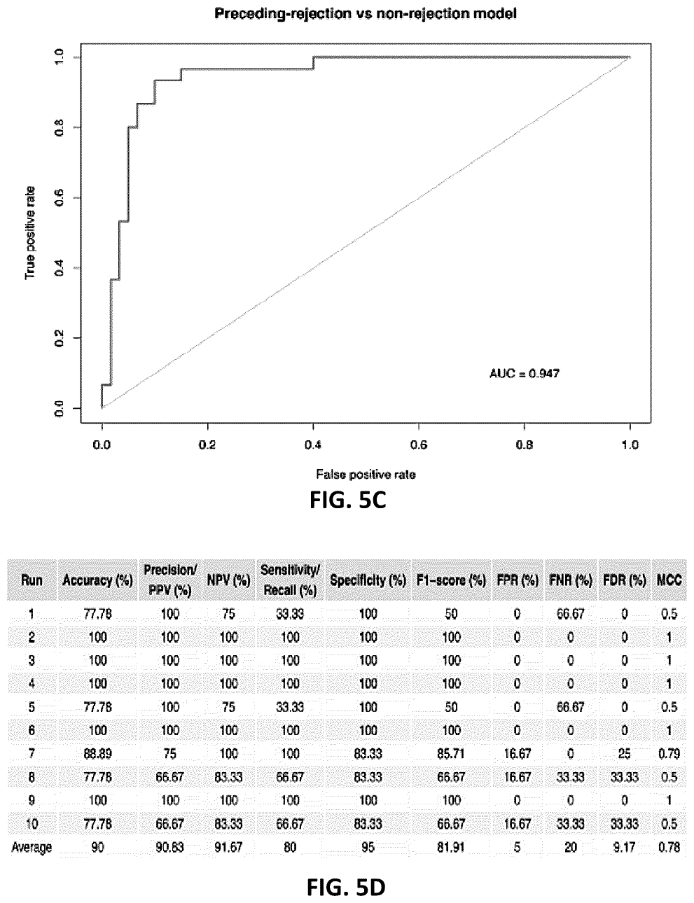

A subset of rejection-associated gene expression exhibited relatively stable high expression in patients that would later experience a rejection event, especially in non-rejection timepoints that immediately preceded a 2R rejection event (FIG. 1A). As such, and without wishing to be bound by theory, it was hypothesized that biopsies adjudicated as non-rejection by traditional histopathological grading exhibit molecular features of early acute rejection as determined by RNA-seq and thus could be prognostic for later acute rejection. In order to test this hypothesis, the set of 0R/1R EMBs was divided into two groups: those immediately preceding a 2R/3R rejection EMB (labeled “preceding-rejection”, PR) and those 0R/1R EMBs not preceding a 2R/3R EMB (labeled “preceding-non-rejection”, PNR). The analysis was also expanded to include the entire transcriptome to avoid excluding any genes that may be specifically associated with the PR group. From this, 528 transcripts were observed to be significantly differentially expressed between the PR and the PNR samples (q<0.01) (FIG. 4 and Table 3). Intriguingly, top genes upregulated preceding rejection included the double homeobox transactivator DUX4 as well as two similar pseudogenes DUX4L19 and DUX4L26 (FIG. 4B). Pathways associated with PR included immune/inflammatory pathways such as cytokine receptor interactions (KEGG q<0.01) as well as genes involved in the inflammatory response (GO, q<0.0022) (FIGS. 5A and B). Of note, these signals were detected across a variety of time intervals between PR and 2R biopsies, with an average time of 88 days, a minimum of nine days and a maximum of 168 days (Table 4).

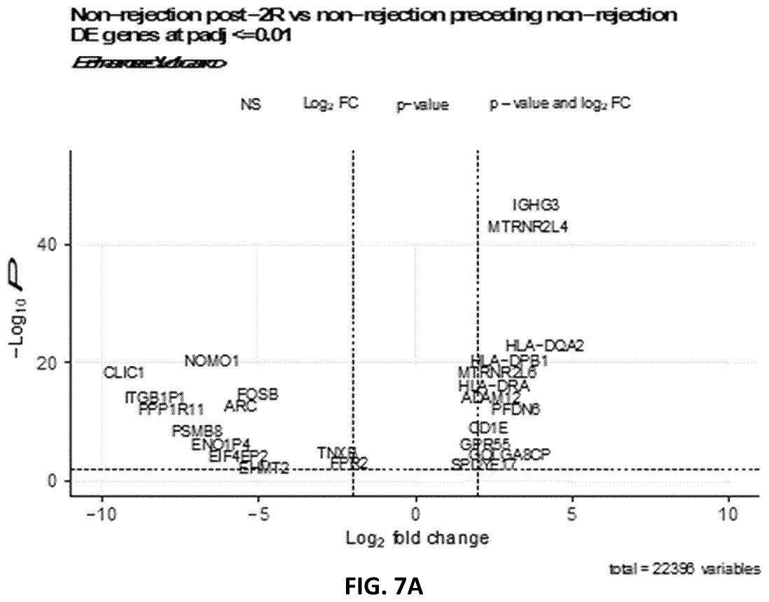

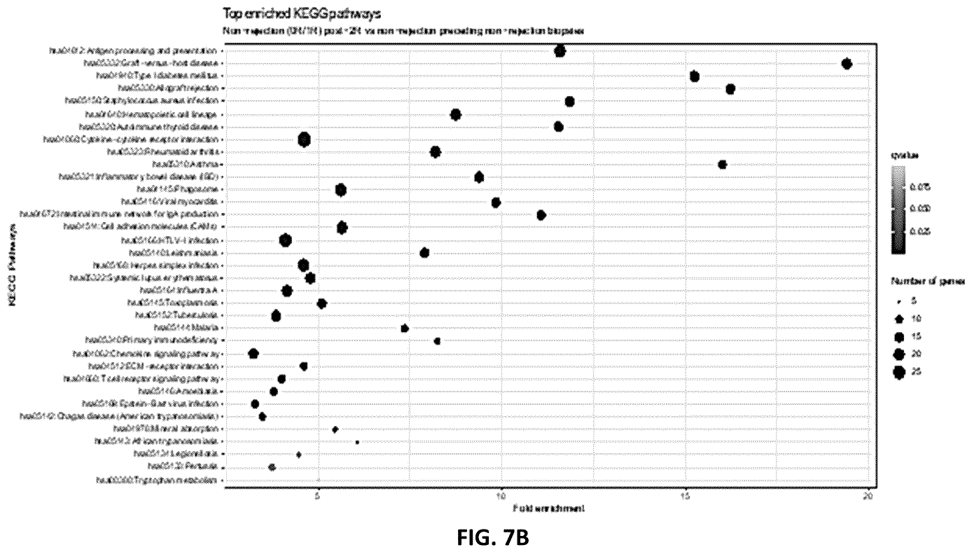

Subsequent studies also examined whether these signals persisted after treatment. Non-rejection biopsies immediately following a treated 2R rejection event were distinctly different from other non-rejection biopsies, and by differential gene and pathway expression exhibited higher expression for many of the same genes and pathways associated with Grade 2R events (FIG. 7). Specifically, pathways associated with antigen presentation and processing as well as graft versus host disease remained the top overexpressed pathways in these non-rejection biopsies. Thus, while histopathological grading does not indicate AR in these biopsies, gene expression profiles indicate residual AR after treatment.

To evaluate whether gene expression profiling of PR samples could act as an early prognostic marker of acute rejection, a classification model of PR versus non-rejection biopsies was generated using a Random Forest approach. Briefly, the set of 0R/1R PR and PNR biopsies were randomly sampled without replacement into 55% training and 45% test sets. A predictor model was built using the 528 differentially expressed genes between the PR and PNR EMB samples. The model was validated through ten rounds of cross validation with resampling of the training and test sets each round, and classification accuracy is shown via ROC plot in FIG. 5C with an AUC of 0.947. Overall statistics show an average classification accuracy of 90% with 90.8% precision (FIG. 5D). The NPV was 91.7%, sensitivity was 80% and specificity was 95%. In conclusion, the classifier exhibits strong performance in assessing whether the biopsy is preceding a rejection or non-rejection timepoint, a capability that may be of significant utility in a clinical setting.

TABLE 4
Time between Grade 2R endomyocardial biopsy
(EMB) and the preceding rejection EMB in
the CTOT-03 Heart Transplant Subjects.

| Participant | Days Preceding 2R Rejection |
|---|---|
| 11015 | 9 |
| 11022 | 58 |
| 11024 | 47 |
| 12003 | 168 |
| 12005 | 165 |
| 12008 | 6 |
| 12013 | 165 |
| Mean | 88.29 |

### Example 4: Selected Discussion

One and five-year survival rates for heart transplant recipients have remained static over the last decade in part due to acute allograft rejection. Although there are differences in reporting accuracies acute rejection is thought to occurs in approximately 30% of heart allograft recipients in the first-year post-transplant alone. A key focus in improving mortality rates is more rapid and accurate AR diagnosis and intervention, however this can be challenging given the invasive nature of EMB as well as the sensitivity and specificity of histopathological evaluation. Other studies have characterized array-based gene expression changes, in a cross-sectional manner, during acute allograft rejection. The studies disclosed herein performed the first large-scale longitudinal characterization which shows that a subset of these transcriptional differences are stable and precede the actual histopathological rejection event in some cases several months before the acute rejection EMB. As such these represent novel putative biomarkers for AR, with higher sensitivity than conventional histopathology. The current gold-standard histological assessment of H&E stains of EMBs is imperfect due to various factors including intra- and inter-pathologist variability in histological classification, or where histological rejection is evident in one EMB for a given recipient but not evident in additional independent EMB from the same timepoint. These limitations impact the sensitivity and specificity and thus the PPV and NPVs of minimally invasive assays such as donor-derived, cell-free (dd-cf)-DNA profiling. Rigorous agnostic assessment and validation of molecular rejection signatures through RNA profiling will thus create a better histological gold standard upon which dd-cf-DNA profiling can be compared against. In a clinical setting, such approaches may ultimately allow for early detection of AR in heart transplant recipients prior to diagnosis by conventional EMB histopathology, thus allowing for more rapid intervention, limiting irreversible graft injury and improving overall outcomes. While evaluation across larger patient cohorts is needed, we hypothesize that integrating gene expression analysis as a standard step in the histopathological evaluation of EMBs will ultimately reduce the number of biopsies needed (i.e., if the prognostic signature is negative, the care team may be able to delay a subsequent biopsy).

In conclusion, the examples of the current disclosure show that RNA-seq reveals a wealth of information regarding the molecular effects of AR in EMBs, most specifically the activation of a multi-faceted immune response involving a wide variety of immune pathways. These signals can be used to accurately classify rejection and non-rejection EMBs and may ultimately be utilized to inform AR diagnosis. Of note, lower levels of these signals already exist in non-rejection biopsies that precede AR diagnosis and can be developed into a highly accurate prognostic classifier that could improve early detection of AR.

### ENUMERATED EMBODIMENTS

The following enumerated embodiments are provided, the numbering of which is not to be construed as designating levels of importance.

Embodiment 1 provides a method for diagnosing and treating acute rejection (AR) of a cardiac transplant tissue in a subject, the method comprising:
contacting a biological sample from the subject with a reagent for assaying the level of at least one biomarker signature nucleic acid,
detecting the amount of the at least one biomarker signature nucleic acid in the biological sample using the reagent,
comparing the level of the at least one biomarker signature nucleic acid from the biological sample to the level of the at least one biomarker signature nucleic acid from a control sample,
determining whether the level of the at least one biomarker signature nucleic acid in the biological sample is an equivalent level, an increased level or a decreased level of the at least one biomarker signature nucleic acid compared to the control sample, wherein an increased level or a decreased level of the at least one biomarker signature nucleic acid in the biological sample relative to the level of the at least one biomarker signature nucleic acid from the control sample indicates that the subject has or is at risk of acute rejection of the cardiac transplant tissue,
and, when the subject is determined to have or be at risk of acute rejection of the cardiac transplant tissue, recommending or providing a treatment to the subject thereby treating the acute rejection.

Embodiment 2 provides the method of claim 1, wherein the at least one biomarker signature nucleic acid is a transcript of a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, ZCCHC12, and any combination thereof.

Embodiment 3 provides the method of claim 1, wherein the at least one biomarker signature nucleic acid is RNA.

Embodiment 4 provides the method of claim 1, wherein the detecting the amount of the at least one biomarker signature nucleic acid in the biological sample is performed using RNA-seq.

Embodiment 5 provides the method of claim 1, wherein the biological sample is an endomyocardial biopsy.

Embodiment 6 provides the method of claim 1, wherein the biological sample is peripheral blood.

Embodiment 7 provides the method of claim 1, wherein the treatment is selected from the group consisting of increasing the dose or frequency of administration of one or more rejection-ameliorating medications, changing to a different rejection-ameliorating medication, and administering one or more medications that suppress the immune system.

Embodiment 8 provides the method of claim 7, wherein the rejection-ameliorating medication is selected from the group consisting of tacrolimus, cyclosporine, prednisone, mycophenolate, azathioprine, sirolimus, everolimus, and any combination thereof.

Embodiment 9 provides the method of claim 1, wherein the subject is a human.

Embodiment 10 provides a method for determining whether a subject is at risk for developing acute rejection (AR) of a cardiac transplant tissue and then treating the subject therefor, the method comprising:
contacting a biological sample from the subject with a reagent for assaying the level of at least one biomarker signature nucleic acid is a transcript of a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, and ZCCHC12,
detecting the amount of the at least one biomarker signature nucleic acid in the biological sample using the reagent,
comparing the level of the at least one biomarker signature nucleic acid from the biological sample to the level of the at least one biomarker signature nucleic acid from a control sample, and
determining whether the level of the at least one biomarker signature nucleic acid in the biological sample is an equivalent level, an increased level or a decreased level of the at least one biomarker signature nucleic acid compared to the control sample, wherein an increased level or a decreased level of the at least one signature nucleic acid in the biological sample relative to the level of the at least one biomarker signature nucleic acid from the control sample indicates that the subject is at risk for developing the acute rejection,
and, when the subject is at risk of developing the acute rejection of a cardiac transplant, recommending or providing a treatment to the subject.

Embodiment 11 provides the method of claim 10, wherein the nucleic acid is RNA.

Embodiment 12 provides the method of claim 10, wherein the detecting of the at least one biomarker signature nucleic acid in the biological sample is performed using RNA-seq.

Embodiment 13 provides the method of claim 10, wherein the biological sample is an endomyocardial biopsy.

Embodiment 14 provides the method of claim 10, wherein the biological sample is peripheral blood.

Embodiment 15 provides the method of claim 10, wherein the treatment is selected from the group consisting of increasing the dose or frequency of administration of one or more rejection-ameliorating medications, changing to a different rejection-ameliorating medication, and administering one or more medications that suppress the immune system.

Embodiment 16 provides the method of claim 15, wherein the rejection-ameliorating medication is selected from the group consisting of tacrolimus, cyclosporine, prednisone, mycophenolate, azathioprine, sirolimus, everolimus, and any combination thereof.

Embodiment 17 provides a composition comprising reagents for assaying the level of at least two biomarker signature nucleic acids useful for diagnosing, predicting, and/or monitoring acute rejection of a cardiac transplant tissue in a sample of a subject, wherein the at least two biomarker signature nucleic acids are transcripts of a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, ZCCHC12, or fragments, or variants thereof.

Embodiment 18 provides the composition of claim 17, wherein the biomarker signature nucleic acids are RNA.

Embodiment 19 provides a kit or assay device for use in diagnosing acute rejection of a cardiac transplant tissue, the kit comprising the composition of claim 17 and instructions for use.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method for diagnosing and treating acute rejection (AR) of a cardiac transplant tissue in a subject, the method comprising:
a. contacting a biological sample from the subject with a reagent for assaying the level of at least one biomarker signature nucleic acid;
b. detecting the amount of the at least one biomarker signature nucleic acid in the biological sample using the reagent;
c. comparing the level of the at least one biomarker signature nucleic acid from the biological sample to the level of the at least one biomarker signature nucleic acid from a control sample;
d. determining whether the level of the at least one biomarker signature nucleic acid in the biological sample is an equivalent level, an increased level or a decreased level of the at least one biomarker signature nucleic acid compared to the control sample, wherein an increased level or a decreased level of the at least one biomarker signature nucleic acid in the biological sample relative to the level of the at least one biomarker signature nucleic acid from the control sample indicates that the subject has or is at risk of acute rejection of the cardiac transplant tissue; and
e. when the subject is determined to have or be at risk of acute rejection of the cardiac transplant tissue, recommending or providing a treatment to the subject thereby treating the acute rejection.

2. The method of claim 1, wherein the at least one biomarker signature nucleic acid is a transcript of a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, ZCCHC12, and any combination thereof.

3. The method of claim 1, wherein the at least one biomarker signature nucleic acid is RNA.

4. The method of claim 1, wherein the detecting the amount of the at least one biomarker signature nucleic acid in the biological sample is performed using RNA-seq.

5. The method of claim 1, wherein the biological sample is an endomyocardial biopsy.

6. The method of claim 1, wherein the biological sample is peripheral blood.

7. The method of claim 1, wherein the treatment is selected from the group consisting of increasing the dose or frequency of administration of one or more rejection-ameliorating medications, changing to a different rejection-ameliorating medication, and administering one or more medications that suppress the immune system.

8. The method of claim 7, wherein the rejection-ameliorating medication is selected from the group consisting of tacrolimus, cyclosporine, prednisone, mycophenolate, azathioprine, sirolimus, everolimus, and any combination thereof.

9. The method of claim 1, wherein the subject is a human.

10. A method for determining whether a subject is at risk for developing acute rejection (AR) of a cardiac transplant tissue and then treating the subject therefor, the method comprising:
a. contacting a biological sample from the subject with a reagent for assaying the level of at least one biomarker signature nucleic acid is a transcript of a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, and ZCCHC12;
b. detecting the amount of the at least one biomarker signature nucleic acid in the biological sample using the reagent;
c. comparing the level of the at least one biomarker signature nucleic acid from the biological sample to the level of the at least one biomarker signature nucleic acid from a control sample;
d. determining whether the level of the at least one biomarker signature nucleic acid in the biological sample is an equivalent level, an increased level or a decreased level of the at least one biomarker signature nucleic acid compared to the control sample, wherein an increased level or a decreased level of the at least one signature nucleic acid in the biological sample relative to the level of the at least one biomarker signature nucleic acid from the control sample indicates that the subject is at risk for developing the acute rejection; and
e. when the subject is at risk of developing the acute rejection of a cardiac transplant, recommending or providing a treatment to the subject.

11. The method of claim 10, wherein the nucleic acid is RNA.

12. The method of claim 10, wherein the detecting of the at least one biomarker signature nucleic acid in the biological sample is performed using RNA-seq.

13. The method of claim 10, wherein the biological sample is an endomyocardial biopsy.

14. The method of claim 10, wherein the biological sample is peripheral blood.

15. The method of claim 10, wherein the treatment is selected from the group consisting of increasing the dose or frequency of administration of one or more rejection-ameliorating medications, changing to a different rejection-ameliorating medication, and administering one or more medications that suppress the immune system.

16. The method of claim 15, wherein the rejection-ameliorating medication is selected from the group consisting of tacrolimus, cyclosporine, prednisone, mycophenolate, azathioprine, sirolimus, everolimus, and any combination thereof.

17. A composition comprising reagents for assaying the level of at least two biomarker signature nucleic acids useful for diagnosing, predicting, and/or monitoring acute rejection of a cardiac transplant tissue in a sample of a subject, wherein the at least two biomarker signature nucleic acids are transcripts of a gene selected from the group consisting of ABHD16A, ADAMDEC1, ADGRG7, ADIPOQ, AICDA, ALB, ANKS1B, ARC, BAG6, BRD2, BTLA, C4A, CCL19, CCR6, CCR8, CD3G, CD5, CD72, CDH4, CELF4, CIDEA, CIDEC, CLC, CLNK, CXCL11, CXCL8, CXCL9, CXCR3, DOC2B, DUX4, DUX4L18, DUX4L19, DUX4L26, EDNRB, FAM220A, FFAR2, FLOT1, FOSB, GNL1, H3P6, HLA-A, HLA-C, HLA-DMB, HLA-DOA, HLA-DPB1, HLA-DQA1, HLA-DQA2, HLA-DQB1, HLA-DRA, HLA-L, HMGN1P14, HS6ST2, HS6ST3, IDO1, IGHA1, IGHG2, IGHG3, IGHG4, IGHV1-2, IGHV1-24, IGHV1-3, IGHV1-45, IGHV3-11, IGHV3-15, IGHV3-20, IGHV3-33, IGHV3-53, IGHV3-7, IGHV3-72, IGHV4-28, IGHV4-31, IGHV4-55, IGKV1-12, IGKV1-13, IGKV1-16, IGKV1-17, IGKV2-28, IGKV2-30, IGKV2D-29, IGKV2D-40, IGKV3D-15, IGLV1-44, IGLV2-14, IGLV2-34, IGLV3-1, IGLV3-10, IGLV3-21, IGLV7-46, ITGB1P1, ITGB1P1, KCNH7, KCNJ10, LAMP3, LEF1, LGALS17A, LHFPL5, LRTM1, LY6G5B, MINAR2, MMP7, MS4A3, MSX1, MTHFD2P1, MTRNR2L1, MTRNR2L11, MTRNR2L4, MTRNR2L8, NA, NAPSB, NOMO1, NPY2R, NWD2, PITX1, PLA2G2A, PLA2G2D, PPM1N, PPP1R11, PPT2, PSG2, PSMB8, RNA5-8SP6, RPS3AP46, RPS6P26, SCUBE1, SLC22A9, SLC2A3P1, SPP1, SRP9P1, TAP1, TIFAB, TM4SF19, TNXB, TRAV6, TRIM26, TSPY3, UBD, UBE2T, XCR1, ZCCHC12, or fragments, or variants thereof.

18. The composition of claim 17, wherein the biomarker signature nucleic acids are RNA.

19. A kit or assay device for use in diagnosing acute rejection of a cardiac transplant tissue, the kit comprising the composition of claim 17 and instructions for use.
